(19) United States
(12) Patent Application Publication
(10) Pub. No.: US 2023/0114049 A1
(43) Pub. Date: Apr. 13, 2023

(54) OPTICAL ARRAY QPCR

(71) Applicant: DeepDiveBio, Inc., San Diego, CA (US)

(72) Inventor: Tei NEWMAN-LEHMAN, San Diego, CA (US)

(21) Appl. No.: 17/794,783

(22) PCT Filed: Jan. 22, 2021
(86) PCT No.: PCT/US2021/014747

§ 371 Date: Jul. 22, 2022

Related U.S. Application Data
(60) Provisional application No. 62/965,766, filed on Jan. 24, 2020.

(51) Int. Cl. B01L 3/00; B01L 7/00; C12Q 1/6851

(52) U.S. Cl. CPC B01L 3/50851; B01L 7/52; C12Q 1/6851; B01L 2300/18; B01L 2300/0636; B01L 2300/0829; B01L 2300/021

(57) ABSTRACT

Provided herein are devices, methods, and systems for polynucleotide synthesis comprising a thermocycler comprising a plurality of individual reaction chambers having a capability to control its own temperature setting. The devices, methods, and systems provided herein further comprise a detection module and a light source that are used to monitor the progress of the polynucleotide synthesis reaction in the individual reaction chambers and the quality and quantity of the synthesized polynucleotides.

### CROSS-REFERENCE

This application claims the benefit of U.S. Provisional Application No. 62/965,766, filed on Jan. 24, 2020, which is incorporated by reference herein in its entirety.

### BACKGROUND

Custom gene synthesis, or polynucleotide synthesis, provides a powerful tool for research in biology and medicine and for various biotechnology applications. Polynucleotide synthesis typically involves building specially designed oligonucleotides, preparing the oligonucleotides and various reagents in a mixture, and assembling the oligonucleotides using a thermocycler. There remains a need for enhancing the quality of the synthesized polynucleotides and reducing the time for the polynucleotide synthesis. The capability to monitor the synthesis in real-time would be helpful to adjust the parameters of the thermocycler as needed.

### SUMMARY

Provided herein are devices, methods, and systems that provides the capability to monitor the progress of the polynucleotide synthesis reactions in real-time and to improve the polynucleotide synthesis through feedback control of the thermocycling devices, systems, and methods. The devices, methods, and systems provided herein allow for a control of a plurality of polynucleotide synthesis reactions through the delivery of precise temperature profiles and optical imaging feedback of the progress of the polynucleotide synthesis reactions. In some embodiments, the device, methods, and systems described herein may control and optimize a plurality of individual polynucleotide synthesis reactions in a highly parallel configuration that may be spatially and/or temporally separated from one another. In some embodiments, a processor with a user interface may the control of one or more components of the device, methods and systems provided herein. Often, the capability to control the individual polynucleotide synthesis reaction in each reaction chamber independently from another reaction may allow the devices, systems and methods described herein to execute a plurality of unique polynucleotide synthesis reactions with different reaction parameters at a given time. In some embodiments, the devices, systems and methods provided herein improves the speed and/or the quality of the polynucleotide synthesis reactions by providing imaging-based feedback. In some instances, the imaging-based feedback may provide information regarding the quantity or amount of polynucleotide synthesis products generated after one or more cycles of thermal fluctuations. In some instances, the imaging-based feedback may provide information to the particular reaction kinetics that may be employed in further deductive conclusions regarding the reaction constituent or products produced.

Aspects of disclosure herein provide a device for thermocycling for polynucleotide synthesis, comprising: (a) a plurality of reaction chambers, each reaction chamber having a top opening and a bottom opening, wherein the top opening and the bottom opening are on opposite ends; (b) a thermoelectric module, wherein the thermoelectric module is thermally coupled to each of the plurality of reaction chambers; (c) a light source, the light source oriented to provide a light beam through the top opening and the bottom opening of the each of the plurality of reaction chambers; and (d) a detection module, the detection module comprising an imaging module the detection module positioned above the plurality of reaction chambers. In some embodiments, the thermoelectric module comprises a plurality of openings, wherein the light source is oriented to provide a light beam through the plurality of openings of the thermoelectric module. In some embodiments, the thermoelectric module is configured to provide a thermal energy to each of the plurality of reaction chambers. In some embodiments, the thermal energy for a first reaction chamber is different from the thermal energy for a second reaction chamber. In some embodiments, each of the plurality of reaction chambers is configured to hold a sample container. In some embodiments, the sample container comprises a lid comprising an identification tag that is detectable by the detection module. In some embodiments, the identification tag comprises one or more of a quick response (QR) code, barcode, alpha-numeric characters, quantum dots, or radio frequency identification (RFID). In some embodiments, the sample container comprises a lid comprising a temperature indicator that is detectable by the detection module. In some embodiments, the temperature indicator comprises a thermochromic label. In some embodiments, the detection module comprises an infrared sensor configured to detect a temperature of the reaction chamber. In some embodiments, a temperature sensor is affixed to a wall of the reaction chamber.

In some embodiments, the light source comprises a light emitting diode (LED). In some embodiments, the LED is a surface mount or through hole LED. In some embodiments, the LED emits ultraviolet (UV), visible, near-infrared (NIR), or infrared (IR) radiative energy, or any combination thereof.

In some embodiments, the detection module further comprises a tunable filter. In some embodiments, the tunable filter is a solid-state tunable filter. In some embodiments, the tunable filter is configured to capture at least IR, visible, and UV wavelengths. In some embodiments, the tunable filter is configured to differentiate between visible and UV light. In some embodiments, the tunable filter comprises a liquid crystal tunable filter, or acousto-optic filter, or any combination thereof. In some embodiments, the detection module is configured to image one or more of visible spectrum, UV light spectrum, or infrared spectrum, or any combination thereof. In some embodiments, the detection module is configured to detect fluorescence, phosphorescence, stimulated emission, auto-fluorescence, fluorescence lifetime or any combination thereof.

In some embodiments the light source comprises a tunable emission of a silicon photonic array, wherein the silicon photonic array comprises a plurality of silicon photonic modules, each silicon photonic module comprising an optical ring resonator. In some embodiments, the optical ring resonator is configured to tune the wavelength of the light source. In some embodiments, the light source is a low coherent diode laser, highly coherent diode laser, super luminescent diode (SLD), vertical cavity surface emitting laser (VCSEL) or any combination thereof. In some embodiments, the tuned light beam is detectable by the detection module. In some embodiments, the tuned light beam has a different wavelength from the wavelength of the light source. In some embodiments, the silicon photonic module further comprises a grating coupler and a beam splitter, wherein the grating coupler and the beam splitter are configured to direct the light source to each silicon photonic module of the plurality of silicon photonic modules. In some embodiments, the optical ring resonator comprises a thermally tunable optical ring resonator. In some embodiments, the silicon photonic module further comprises an integrated heater, wherein the integrated heater is configured to provide a thermal energy to one of the plurality of reaction chambers, and wherein the thermal optical ring resonator is configured to be tuned by the thermal energy.

In some embodiments, the device further comprises a power source, wherein the power source provides power to the thermoelectric module. In some embodiments, the power ranges from about 0W to about 5W.

In some embodiments, the device is connected to a processor, wherein the processor is electrically coupled to the thermoelectric module, the light source, and the detection module. In some embodiments, the processor analyzes a detected light beam emitted from the sample in the sample container.

In some embodiments the sensor comprises one or more of a two-dimensional CMOS, two-dimensional CCD, linear array CMOS, linear array CCD, avalanche photodiode, single photodiode, or balanced photodetector sensor, or any combination thereof.

Aspects of the disclosure herein provide a method of monitoring thermocycling for polynucleotide synthesis of a sample, the method comprising: (a) receiving a sample container holding a sample into one of a plurality of reaction chambers of a thermocycler; (b) detecting by a detection module an identification tag on the sample container, wherein the identification tag comprises information of a thermocycling protocol; (c) applying the thermocycling protocol to the reaction chamber by a thermoelectric module of the thermocycler, where the thermoelectric module delivers thermal energy to the reaction chamber and the sample container according to the thermocycling protocol; (d) illuminating the sample container in the reaction chamber with a light beam from a light source; and (e) detecting by the detection module an output light beam emitted from the sample container, wherein the output light beam comprises the light beam that passed through the sample. In some embodiments, the sample in the sample container undergoes polynucleotide synthesis. In some embodiments, detecting by a detection module an identification tag comprises identifying the identification tag by a system controller and matching the identification tag to the thermocycling protocol in a database. In some embodiments, the identification tag comprises one or more of a quick response (QR) code, barcode, alpha-numeric characters, quantum dots, or radio frequency identification (RFID), or any combination thereof. In some embodiments, the method comprises repeating the steps of illuminating, detecting, and analyzing during polynucleotide synthesis.

In some embodiments, the detection module comprises a sensor. In some embodiments the sensor comprises one or more of a two-dimensional CMOS, two-dimensional CCD, linear array CMOS, linear array CCD, avalanche photodiode, single photodiode, balanced photodetector sensor, or any combination thereof. In some embodiments, the detection module comprises a tunable filter, wherein the thermocycler comprises a system controller that is configured to adjust the tunable filter to a target wavelength spectra. In some embodiments, the detection module is configured to image visible and UV spectra. In some embodiments, the detection module is configured to detect fluorescence, phosphorescence, stimulated emission, auto-fluorescence, fluorescence lifetime emission or any combination thereof. In some embodiments, the target wavelength spectra comprise spectra in the ultraviolet, visible, near-infrared, or infrared, or any combination thereof. In some embodiments, the system controller adjusts the tunable filter to a target wavelength range based on the thermocycling protocol. In some embodiments, the method further comprises adjusting the tunable filter by the system controller to visible wavelengths to image the identification tag. In some embodiments, the method further comprises adjusting the tunable filter by the system controller to a target wavelength to image a thermochromic label on the sample container. In some embodiments, the method further comprises adjusting the tunable filter by the system controller to a target near-infrared or infrared spectra to measure a temperature of the reaction chamber. In some embodiments, the method further comprises applying the thermocycling protocol to the reaction chamber by the thermoelectric module until the detection module detects a color change on the thermochromic label indicating a target temperature. In some embodiments, the method further comprises applying the thermocycling protocol to the reaction chamber by the thermoelectric module until the detection module detects a spectra of near-infrared or infrared emitted by the reaction chamber indicating a target temperature. In some embodiments, the method further comprises adjusting the tunable filter by the system controller to a target output wavelength to detect an output light beam from the sample. In some embodiments, the target output wavelength comprises wavelength spectra in the ultraviolet, visible, near-infrared, infrared or any combination thereof. In some embodiments, the tunable filter is a solid-state tunable filter. In some embodiments, the tunable filter comprises a liquid crystal tunable filter, or acousto-optic filter, or any combination thereof. In some embodiments, the tunable filter is configured to capture at least IR, visible, and UV wavelengths. In some embodiments, the tunable filter is configured to differentiate between visible and UV light

In some embodiments, the sample comprises a fluorescent label. In some embodiments, the sample comprises a fluorescently labeled nucleotide probe. In some embodiments, the output light beam comprises one or more of a fluorescence, phosphorescence, stimulated emission, auto-fluorescence, or fluorescence lifetime emission, or any combination thereof.

In some embodiments, an intensity of the output light beam is correlated to the amount of the synthesized polynucleotide in the sample. In some embodiments, a wavelength of the output light beam is correlated to the amount of the synthesized polynucleotide in the sample.

In some embodiments the thermocycling protocol comprises a set of target temperatures for the reaction chamber to reach during the polynucleotide synthesis.

In some embodiments, the thermoelectric module comprises a plurality of openings, wherein the light source is oriented to provide a light beam through the plurality of openings of the thermoelectric module. In some embodiments, the thermoelectric module is configured to provide a thermal energy to each of the plurality of reaction chambers. In some embodiments, the thermal energy for a first reaction chamber is different from the thermal energy for a second reaction chamber.

In some embodiments, the light source comprises a light emitting diode (LED). In some embodiments, the LED is a surface mount or through hole LED. In some embodiments, the LED emits ultraviolet (UV), visible, near-infrared (NIR), or infrared (IR) radiative energy, or any combination thereof. In some embodiments, the light source comprises a low coherent diode laser, highly coherent diode laser, super luminescent diode (SLD), vertical cavity surface emitting laser (VCSEL) or any combination thereof.

In some embodiments, the thermocycler further comprises a silicon photonic array, the silicon photonic array comprising a plurality of silicon photonic modules, each silicon photonic module comprising an optical ring resonator. In some embodiments, the optical ring resonator is configured to tune the wavelength of the light beam. In some embodiments the tuned light beam is detectable by the detection module. In some embodiments, the tuned light beam has a different wavelength from the wavelength of the light source. In some embodiments, the silicon photonic module further comprises a grating coupler and a beam splitter, wherein the grating coupler and the beam splitter are configured to direct the light beam from the light source to each of the plurality of optical ring resonators. In some embodiments, the optical ring resonator comprises a thermally tunable optical ring resonator. In some embodiments, the silicon photonic module further comprises an integrated heater, wherein the integrated heater is configured to provide a thermal energy to one of the plurality of thermally tunable optical ring resonators. In some embodiments, the thermal energy provided to one of the plurality of thermally tunable optical ring resonators tunes the light beam. In some embodiments, the thermocycler comprises a system controller that is electrically coupled to the thermoelectric module, the light source, and the detection module.

In some embodiments, the thermocycler comprises a power source, wherein the power source provides power to the thermoelectric module. In some embodiments, the power ranges from about 0W to about 5W.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a thermoelectric PCBA (“TEC board”) with a LED and thermoelectric module.

FIG. 2 shows an assembly of the thermoelectric module(s) over the LED(s), where the thermoelectric modules are installed on the TEC board after positioning and soldering the LED(s) onto the TEC board.

FIG. 3A-3B illustrate the position and orientation of an LED light source with respect to a thermoelectric module. Additionally, an isolated thermoelectric module is shown schematically to illustrate exemplary size of an inner and outer diameter of the isolated thermoelectric module as well as the solder tabs used to position and center the thermoelectric module.

FIG. 4 shows a cross section of the thermoelectric PCBA, thermoelectric module, and LED illustrating the light path of the LED may be through a center cutout of the thermoelectric module.

FIGS. 5A-5B shows a top-down and cross-sectional view of the thermoelectric PCBA comprising the LED light source assembly and cross-sectional view of the thermoelectric well as a cross section comprising the heatsink, TEC board, silicon photonic device, reaction chamber, and sample vial.

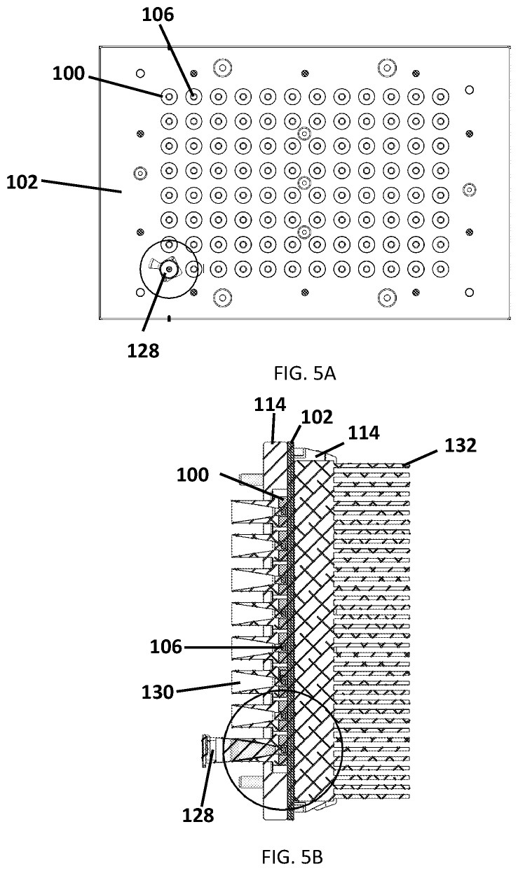

FIGS. 6A-6B illustrate a sample vial placed into a reaction chamber and the structure of the reaction chamber with a circular perforation at the bottom of the chamber permitting collimated light from the LED positioned below the chamber to couple into the sample vial.

FIG. 7 shows the arrangement of the components of a thermocycler particularly the detection module sensor and tunable filter in optical communication with the thermoelectric module, sample vial, reaction chamber and LED used to illuminate the sample vial.

FIGS. 8A-8B illustrate a macroscopic and enlarged perspective of the silicon photonic comprised of a plurality of optical ring resonator.

FIGS. 9A-9C illustrate the spatial relationship between a diode light source, silicon photonic device, and the thermoelectric module comprising the thermoelectric PCBA, thermoelectric modules, reaction chamber, heatsink and sample vials. Additionally, a detailed schematic of the silicon photonic device grating coupler and beam splitter receiving and distributing the light source are schematically represented.

FIGS. 10A-10B illustrate the configuration of the silicon photonic device emitted light path with respect to the thermoelectric PCBA, thermoelectric module, reaction chamber, sample vial and heatsink. In addition, an enlarged schematic of the light path emitted by the silicon photonic device is shown.

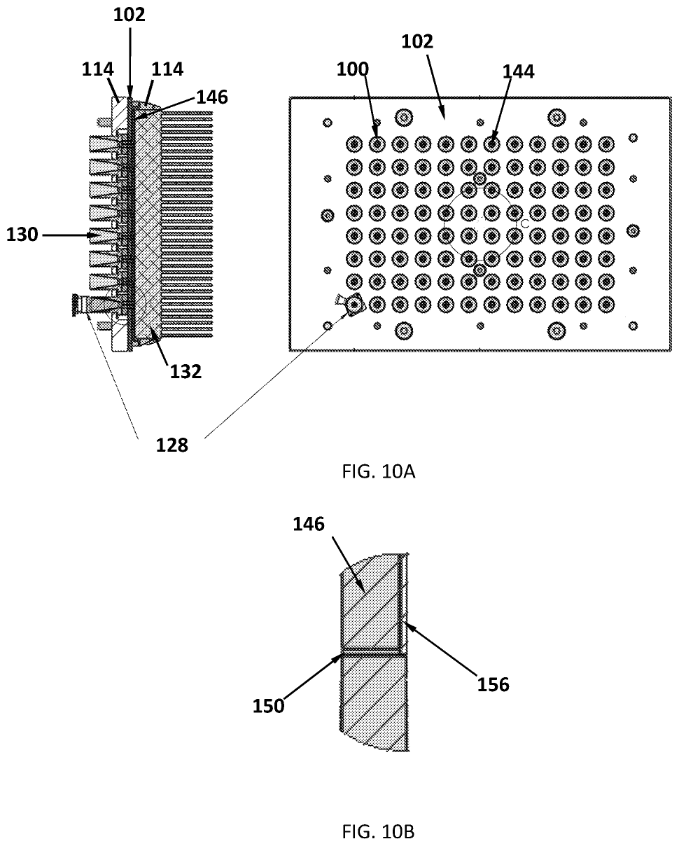

FIG. 11 shows a workflow diagram for monitoring a plurality of polynucleotide synthesis with a thermocycler device.

FIG. 12 illustrates a system schematic of the thermocycler device configured for off-line or cloud based operation.

### DETAILED DESCRIPTION

Custom gene synthesis, or custom polynucleotide synthesis, provides a powerful tool for research in biology, medicine, and for various biotechnology applications. Gene synthesis, or polynucleotide synthesis, typically involves synthesizing specially designed oligonucleotides, preparing the oligonucleotides and various reagents in a mixture, and assembling the oligonucleotides using a thermocycler to generate a polynucleotide or a gene. Once the mixture comprising the oligonucleotides and various reagents is placed inside the thermocycler, the mixture is exposed to a temperature profile that is iterated several times. An example of such polynucleotide synthesis is a Gibson assembly. One hurdle in polynucleotide synthesis is that assembling complex polynucleotides from oligonucleotides, or nucleic acid molecules, often can be error prone. There is a need for improved devices, systems, and methods for custom polynucleotide synthesis, including improving the quality of the synthesized polynucleotide and reducing the time required to generate the polynucleotides.

The quality of a polynucleotide generated by the polynucleotide synthesis may depend on a number of factors, including but not limited to production of the oligonucleotides, choice of reagents, concentrations of reagents or oligonucleotides, or temperature profiles that the mixture is subject to during thermocycling. Usually, the quality of the synthesized polynucleotide, or the success or failure of the polynucleotide synthesis reaction, may be assessed by running the reacted mixture on an agarose gel or cloning the product into a plasmid and sequencing it, which can be time consuming. As such, the capability to assess the quantity and/or quality of the synthesized polynucleotide in real-time or close to real-time would be advantageous. In some embodiments, monitoring the polynucleotide synthesis reaction may provide opportunities to adjust the reaction conditions, including but not limited to reaction chamber temperature or lid temperature.

Often, the conditions for the polynucleotide synthesis reactions can vary and introduce sources of error or decrease in quality or quantity of synthesized polynucleotides. In some cases, polynucleotide synthesis reaction may need to be repeated with adjustments in the synthesis conditions. The adjusted conditions sometimes comprise at least one of adding a new reagent, changing a concentration of a reagent in the mixture, or changing the temperature conditions that the mixture is subjected to by the thermocycler. Often, the adjustments to the polynucleotide synthesis processes comprises changing the temperature and/or the length of time at a given temperature that the mixture is subject to by the thermocycler. Many commercially available thermocyclers are limited in the temperature conditions that it can apply within a single reaction cycle across a multi-well plate comprising a plurality of reaction chambers. This limits the number of simultaneous polynucleotide synthesis process conditions that can be tested at one time.

In the chaotic laboratory environment, it may be difficult to keep track of the numerous input factors that go into various experiments. These input factors are critical to assessing trends in experiments and can be a tedious process for a user to maintain a proper record of the detailed protocol for each experiment, especially in a research and development environment where intuitive decisions are highly valued. Experimental details can be recorded in laboratory notebooks, or digitally using spreadsheets or other software programs. However, in a lab environment, freeing one's hands from a pipette, or removing a glove to record experimental details may delayed until the end of the experiment. This may leave room for error in the record of the experiment. As such, the capability to automatically detect and record reaction conditions can be highly valuable. In some embodiments, a detection module can detect the unique identifier, such as a QR code or barcode, on the lid of a sample vial and record the corresponding reaction conditions. In some embodiments, the unique identifier may comprise a temperature protocol information, and the detection of the unique identifier may provide the temperature protocol information to the thermocycler to apply the temperature protocol to the specific reaction chamber holding the sample vial.

The devices, methods, and systems provided herein may provide various advantages. In some embodiments, the devices, methods, and systems provided herein may improve the quality and/or quantity of the synthesized polynucleotides, improve the temperature control of the individual reaction chambers and sample vials, improve throughput, reduce cost, and provide a simple user interface to facilitate collaboration in polynucleotide synthesis. The devices, methods, and systems may comprise a thermocycler. In some embodiments, the system comprises a device connected to a processor. In some embodiments, the system comprises a machine learning polynucleotide synthesis algorithm. In some cases. the thermocycler may comprise a plurality of individual reaction chambers, where the temperature setting of an individual reaction chamber may be controlled independently from the temperature setting of another individual reaction chamber. The devices, methods, and systems provided herein can control parameters of reaction chambers and is small, modular, and easy to maintain. The devices, methods, and systems provided herein may be compatible with laboratory automation, mobile devices, and laboratory information management software (LIMS) to improve throughput, synthesis quality, and reproducibility. Such devices, methods, and systems may improve quality of the polynucleotide product and success of the polynucleotide synthesis reactions by monitoring the progress of the polynucleotide synthesis reactions and providing feedback to adjust parameters of the reaction chambers and/or sample vial accordingly. The devices, methods, and systems provided herein may improve the throughput by running multiple protocols with different steps in parallel and using a modular architecture that is scalable. The devices, methods, and systems provided herein may reduce costs by requiring fewer instruments where each reaction chamber serves as an individual thermocycler device with independent functionality from another well.

### Thermocycler Device

Aspects of the present disclosure may comprise a thermocycler device for the thermocycling of polynucleotide synthesis configured to improve throughput and yield of one or more polynucleotide synthesis reactions. The thermocycler device described herein may comprise a one or more reaction chambers 130, one or more thermoelectric modules 100, one or more light sources 106 and 144 and one or more detection modules 141. The polynucleotide synthesis reaction of the thermocycler device may occur in one or more sample vials 128, which may be placed into a reaction chamber. In some embodiments, the sample vials hold oligonucleotides and reagents for the polynucleotide synthesis reaction. In some embodiments, the thermoelectric module generates and provides a thermal energy to the reaction chamber. In some embodiments, the thermal energy delivered to the reaction chamber changes the temperature of the sample vial and the contents of the sample vial. In some embodiments, the thermoelectric module comprises a circuit patch comprising a heating element. In some embodiments, the circuit patch is placed on the lid of the sample vial. In some embodiments, the circuit patch is a covering film covering over the sample vial. In some embodiments, the circuit patch comprises conductive contacts that is configured to electrically interface with the device. In some embodiments, the circuit patch comprises conductive contacts that is configured to interface with the contacts on a printed circuit board (PCB) of the device. In some embodiments, the device is configured to control each heating element for a sample vial individually. In some embodiments, the device is configured to control each heating element for one sample vial independently from another heating element for another sample vial. In some embodiments, the device is configured to control a set of heating elements for a set of sample vials independently from another set of heating elements for another set of sample vials. In some embodiments, the heating element on the lid reduces the condensation that forms on the lid. In some embodiments, the reduction in condensation improves the transmission of the light beam from the light source through the sample vial. In some embodiments, the reduction in condensation improves detection of the emitted light from the sample vial. In some embodiments, the reaction chamber comprises a hole through the length of the chamber to allow a light source to transmit a light beam through the length of the reaction chamber. In some embodiments, the light beam from the light source is transmitted through the length of the sample vial. In some embodiments, one or more reagents of in the sample vial emit fluorescence when excited by the light beam.

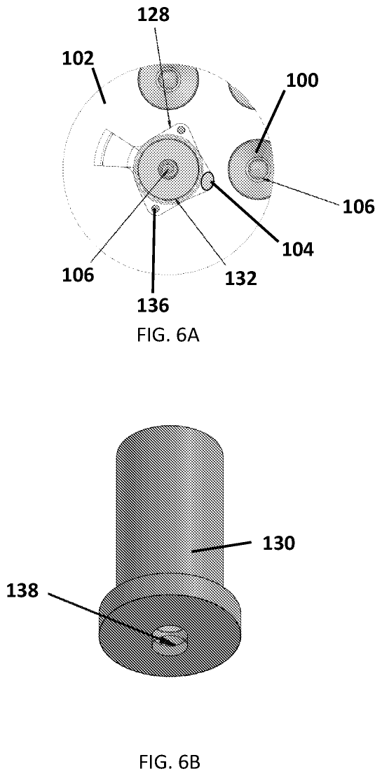

In some instances, the one or more sample vials 128 may include but are not limited to single-use tubes, Eppendorf® tubes, PCR tubes, multi-well strips, multi-well plate, or any equivalent thereof. In some instances, the one or more sample vials 128 may comprise a lid having a thermal colorimetric indicator 132. In some cases, the thermal colorimetric indicator may comprise a thermochromic ink that may change color based on the temperature of the lid. In some instances, the thermochromic ink may be printed, or adhered to a lid of the one or more sample vials 128 using an adhesive. Alternatively, the thermochromic ink may be added to the polynucleotide synthesis reagents directly. In some instances, the lid of the one or more sample vial 128 may comprise a region absent of thermochromic ink where the emitted light of sample by one or more light sources 106 may be visualized, as seen in FIG. 6A. In some cases, the one or more sample vial may comprise one or more heat transducing members 136 thermally coupled to the one or more thermoelectric modules 100. In some cases, the one or more sample vials 128 may comprise an identifier 104 (FIG. 6A) comprising a quick response (QR) code, barcode, alpha-numeric characters, quantum dots, or radio frequency identification (RFID), or any combination thereof. In some instances, the sample vial identifier may indicate to a processor the temperature profile for a polynucleotide synthesis reaction and excitation wavelength of the light source.

The thermocycler device may be configured to execute one or more polynucleotide synthesis reaction simultaneously. Alternatively or in combination, the thermocycler device may be configured for polynucleotide synthesis where one or more polynucleotide synthesis reactions may begin and end at varying start and end times. In some instances, the thermocycler device may interface with automatic liquid handling and sample vial handling robotic systems. Alternatively or in combination, the device may interface with a human operator that may place one or more sample vials in the one or more reaction chambers of the device. The device may comprise a user interface and processor that may program and control the functionality of the device to execute one or more polynucleotide synthesis reactions simultaneously, asynchronously or any combination thereof.

### Thermoelectric Module

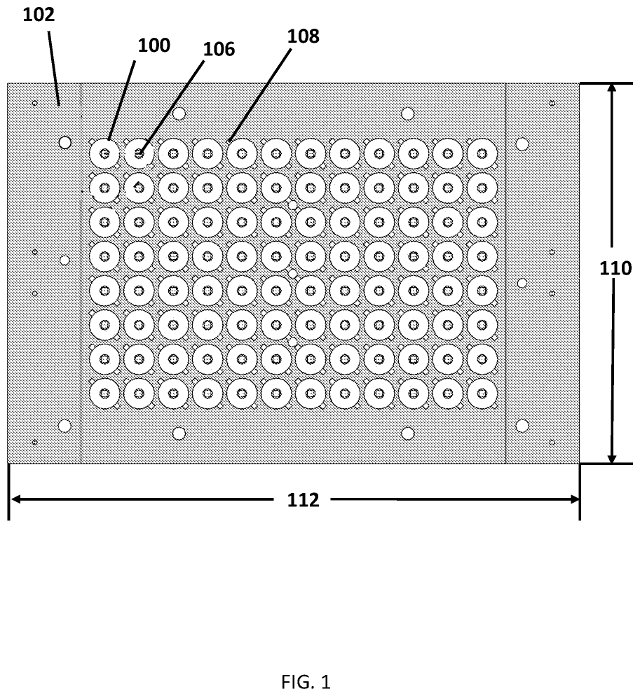

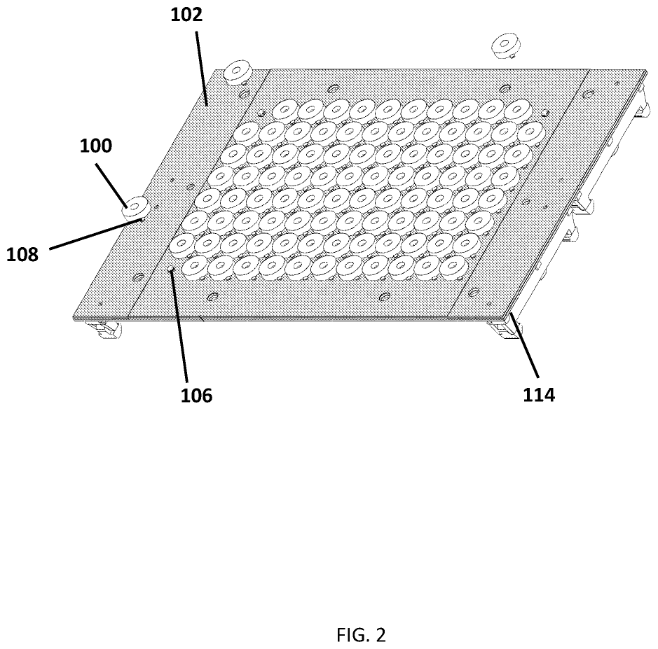

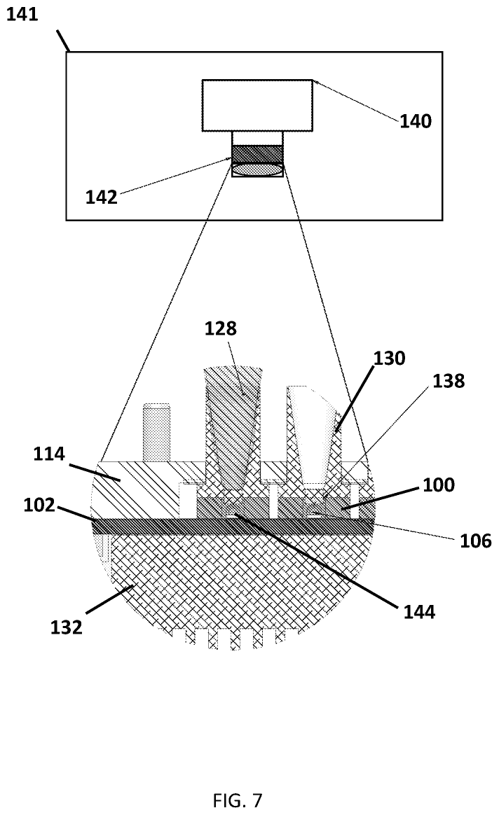

The thermocycler device may comprise one or more thermoelectric module 100, as can be seen in FIG. 1 that may be thermally coupled to a reaction chamber 130, seen in FIG. 7. In some instances, the one or more thermoelectric modules may be electrically coupled to a thermoelectric PCBA 102. In some cases, the one or more thermoelectric modules 100 may be patterned on the thermoelectric PCBA 102. In some instances, the pattern may be linear, as seen in FIG. 1, circular or any combination thereof. In some instances, the thermocycler device may comprise one or more light sources 106. In some instances, the thermoelectric module 100 may comprise solder tabs 108 that may be electrically and mechanically coupled to a thermoelectric printed circuit board assembly (PCBA) 102, as seen in FIG. 1 and FIG. 2. In some embodiments, the device comprises at least 96 thermoelectric modules. In some embodiments, the device comprises 96, 384, or 1536 thermoelectric modules. In some embodiments, the thermoelectric modules are spaced and located similarly to a multi-well plate. In some cases, the reaction chamber may be mechanically and thermally coupled to the thermoelectric modules 100 via a well holding fixture 114, as seen in FIG. 2.

In some cases, the thermoelectric PCBA 102 may comprise a length 112 and a width 110, as seen in FIG. 1. In some instances, the width 110 of the thermoelectric PCBA 102 may be about 50 mm to about 150 mm. In some instances, the width 110 of the thermoelectric PCBA 102 may be about 50 mm to about 60 mm, about 50 mm to about 70 mm, about 50 mm to about 80 mm, about 50 mm to about 90 mm, about 50 mm to about 100 mm, about 50 mm to about 110 mm, about 50 mm to about 120 mm, about 50 mm to about 130 mm, about 50 mm to about 140 mm, about 50 mm to about 150 mm, about 60 mm to about 70 mm, about 60 mm to about 80 mm, about 60 mm to about 90 mm, about 60 mm to about 100 mm, about 60 mm to about 110 mm, about 60 mm to about 120 mm, about 60 mm to about 130 mm, about 60 mm to about 140 mm, about 60 mm to about 150 mm, about 70 mm to about 80 mm, about 70 mm to about 90 mm, about 70 mm to about 100 mm, about 70 mm to about 110 mm, about 70 mm to about 120 mm, about 70 mm to about 130 mm, about 70 mm to about 140 mm, about 70 mm to about 150 mm, about 80 mm to about 90 mm, about 80 mm to about 100 mm, about 80 mm to about 110 mm, about 80 mm to about 120 mm, about 80 mm to about 130 mm, about 80 mm to about 140 mm, about 80 mm to about 150 mm, about 90 mm to about 100 mm, about 90 mm to about 110 mm, about 90 mm to about 120 mm, about 90 mm to about 130 mm, about 90 mm to about 140 mm, about 90 mm to about 150 mm, about 100 mm to about 110 mm, about 100 mm to about 120 mm, about 100 mm to about 130 mm, about 100 mm to about 140 mm, about 100 mm to about 150 mm, about 110 mm to about 120 mm, about 110 mm to about 130 mm, about 110 mm to about 140 mm, about 110 mm to about 150 mm, about 120 mm to about 130 mm, about 120 mm to about 140 mm, about 120 mm to about 150 mm, about 130 mm to about 140 mm, about 130 mm to about 150 mm, or about 140 mm to about 150 mm. In some instances, the width 110 of the thermoelectric PCBA 102 may be about 50 mm, about 60 mm, about 70 mm, about 80 mm, about 90 mm, about 100 mm, about 110 mm, about 120 mm, about 130 mm, about 140 mm, or about 150 mm. In some instances, the width 110 may be at least about 50 mm, about 60 mm, about 70 mm, about 80 mm, about 90 mm, about 100 mm, about 110 mm, about 120 mm, about 130 mm, or about 140 mm. In some instances, the width 110 of the thermoelectric PCBA 102 may be at most about 60 mm, about 70 mm, about 80 mm, about 90 mm, about 100 mm, about 110 mm, about 120 mm, about 130 mm, about 140 mm, or about 150 mm.

In some cases, the length 120 of the thermoelectric PCBA 102 may be about 100 mm to about 200 mm. In some cases, the length 120 of the thermoelectric PCBA 102 may be about 100 mm to about 110 mm, about 100 mm to about 120 mm, about 100 mm to about 130 mm, about 100 mm to about 140 mm, about 100 mm to about 150 mm, about 100 mm to about 160 mm, about 100 mm to about 170 mm, about 100 mm to about 180 mm, about 100 mm to about 190 mm, about 100 mm to about 200 mm, about 110 mm to about 120 mm, about 110 mm to about 130 mm, about 110 mm to about 140 mm, about 110 mm to about 150 mm, about 110 mm to about 160 mm, about 110 mm to about 170 mm, about 110 mm to about 180 mm, about 110 mm to about 190 mm, about 110 mm to about 200 mm, about 120 mm to about 130 mm, about 120 mm to about 140 mm, about 120 mm to about 150 mm, about 120 mm to about 160 mm, about 120 mm to about 170 mm, about 120 mm to about 180 mm, about 120 mm to about 190 mm, about 120 mm to about 200 mm, about 130 mm to about 140 mm, about 130 mm to about 150 mm, about 130 mm to about 160 mm, about 130 mm to about 170 mm, about 130 mm to about 180 mm, about 130 mm to about 190 mm, about 130 mm to about 200 mm, about 140 mm to about 150 mm, about 140 mm to about 160 mm, about 140 mm to about 170 mm, about 140 mm to about 180 mm, about 140 mm to about 190 mm, about 140 mm to about 200 mm, about 150 mm to about 160 mm, about 150 mm to about 170 mm, about 150 mm to about 180 mm, about 150 mm to about 190 mm, about 150 mm to about 200 mm, about 160 mm to about 170 mm, about 160 mm to about 180 mm, about 160 mm to about 190 mm, about 160 mm to about 200 mm, about 170 mm to about 180 mm, about 170 mm to about 190 mm, about 170 mm to about 200 mm, about 180 mm to about 190 mm, about 180 mm to about 200 mm, or about 190 mm to about 200 mm. In some cases, the length 120 of the thermoelectric PCBA 102 may be about 100 mm, about 110 mm, about 120 mm, about 130 mm, about 140 mm, about 150 mm, about 160 mm, about 170 mm, about 180 mm, about 190 mm, or about 200 mm. In some cases, the length 120 of the thermoelectric PCBA 102 may be at least about 100 mm, about 110 mm, about 120 mm, about 130 mm, about 140 mm, about 150 mm, about 160 mm, about 170 mm, about 180 mm, or about 190 mm. In some cases, the length 120 of the thermoelectric PCBA 102 may be at most about 110 mm, about 120 mm, about 130 mm, about 140 mm, about 150 mm, about 160 mm, about 170 mm, about 180 mm, about 190 mm, or about 200 mm.

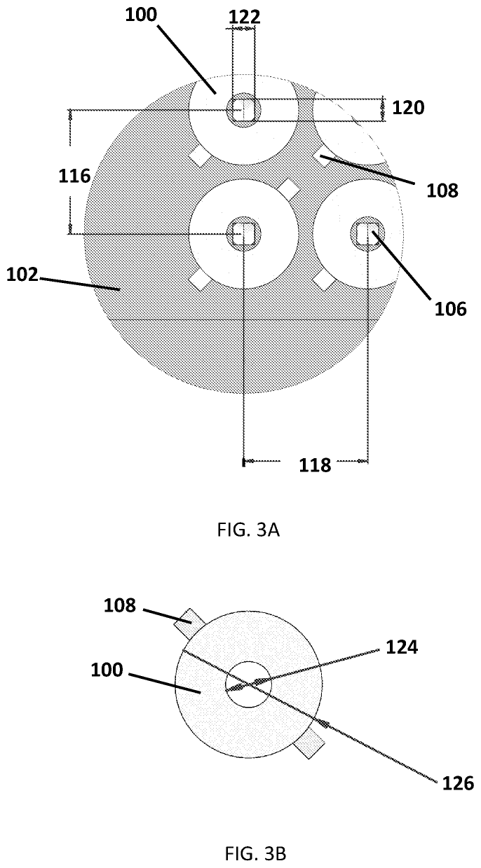

In some cases, the linear pattern of the one or more thermoelectric modules 100, as seen in FIG. 3A, may comprise a center spacing length 118 and width 116. In some instances, the center spacing of the one or more thermoelectric modules 100 length 118 may be about 9 mm to about 15 mm. In some instances, the center spacing of the one or more thermoelectric modules 100 in the length axis 118 may be about 9 mm to about 10 mm, about 9 mm to about 11 mm, about 9 mm to about 12 mm, about 9 mm to about 13 mm, about 9 mm to about 14 mm, about 9 mm to about 15 mm, about 10 mm to about 11 mm, about 10 mm to about 12 mm, about 10 mm to about 13 mm, about 10 mm to about 14 mm, about 10 mm to about 15 mm, about 11 mm to about 12 mm, about 11 mm to about 13 mm, about 11 mm to about 14 mm, about 11 mm to about 15 mm, about 12 mm to about 13 mm, about 12 mm to about 14 mm, about 12 mm to about 15 mm, about 13 mm to about 14 mm, about 13 mm to about 15 mm, or about 14 mm to about 15 mm. In some instances, the center spacing of the one or more thermoelectric modules 100 length 118 may be about 9 mm, about 10 mm, about 11 mm, about 12 mm, about 13 mm, about 14 mm, or about 15 mm. In some instances, the center spacing of the one or more thermoelectric modules 100 length 118 may be at least about 9 mm, about 10 mm, about 11 mm, about 12 mm, about 13 mm, or about 14 mm. In some instances, the center spacing of the one or more thermoelectric modules 100 length 118 may be at most about 10 mm, about 11 mm, about 12 mm, about 13 mm, about 14 mm, or about 15 mm.

In some cases, the center spacing of the one or more thermoelectric modules 100 width 116 may be about 9 mm to about 15 mm. In some cases, the center spacing of the one or more thermoelectric modules 100 width 116 may be about 9 mm to about 10 mm, about 9 mm to about 11 mm, about 9 mm to about 12 mm, about 9 mm to about 13 mm, about 9 mm to about 14 mm, about 9 mm to about 15 mm, about 10 mm to about 11 mm, about 10 mm to about 12 mm, about 10 mm to about 13 mm, about 10 mm to about 14 mm, about 10 mm to about 15 mm, about 11 mm to about 12 mm, about 11 mm to about 13 mm, about 11 mm to about 14 mm, about 11 mm to about 15 mm, about 12 mm to about 13 mm, about 12 mm to about 14 mm, about 12 mm to about 15 mm, about 13 mm to about 14 mm, about 13 mm to about 15 mm, or about 14 mm to about 15 mm. In some cases, the center spacing of the one or more thermoelectric modules 100 width 116 may be about 9 mm, about 10 mm, about 11 mm, about 12 mm, about 13 mm, about 14 mm, or about 15 mm. In some cases, the center spacing of the one or more thermoelectric modules 100 in the width axis 116 may be at least about 9 mm, about 10 mm, about 11 mm, about 12 mm, about 13 mm, or about 14 mm. In some cases, the center spacing of the one or more thermoelectric modules 100 width 116 may be at most about 10 mm, about 11 mm, about 12 mm, about 13 mm, about 14 mm, or about 15 mm.

The thermoelectric module may comprise a circular, square, triangular, hexagonal, or polygonal geometry. In some cases, the circular thermoelectric geometry may comprise an inner 124 and outer diameter 126, as seen in FIG. 3B. In some instances, the circular thermoelectric inner diameter 124 may be about 0.5 mm to about 5.5 mm. In some instances, the circular geometry thermoelectric inner 124 diameter may be about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 3.5 mm, about 0.5 mm to about 4 mm, about 0.5 mm to about 4.5 mm, about 0.5 mm to about 5 mm, about 0.5 mm to about 5.5 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 4.5 mm, about 1 mm to about 5 mm, about 1 mm to about 5.5 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 1.5 mm to about 4.5 mm, about 1.5 mm to about 5 mm, about 1.5 mm to about 5.5 mm, about 2 mm to about 2.5 mm, about 2 mm to about 3 mm, about 2 mm to about 3.5 mm, about 2 mm to about 4 mm, about 2 mm to about 4.5 mm, about 2 mm to about 5 mm, about 2 mm to about 5.5 mm, about 2.5 mm to about 3 mm, about 2.5 mm to about 3.5 mm, about 2.5 mm to about 4 mm, about 2.5 mm to about 4.5 mm, about 2.5 mm to about 5 mm, about 2.5 mm to about 5.5 mm, about 3 mm to about 3.5 mm, about 3 mm to about 4 mm, about 3 mm to about 4.5 mm, about 3 mm to about 5 mm, about 3 mm to about 5.5 mm, about 3.5 mm to about 4 mm, about 3.5 mm to about 4.5 mm, about 3.5 mm to about 5 mm, about 3.5 mm to about 5.5 mm, about 4 mm to about 4.5 mm, about 4 mm to about 5 mm, about 4 mm to about 5.5 mm, about 4.5 mm to about 5 mm, about 4.5 mm to about 5.5 mm, or about 5 mm to about 5.5 mm. In some instances, the circular geometry thermoelectric inner diameter 124 may be about 0.5 mm, about 1 mm, about 1.5 mm, about 2 mm, about 2.5 mm, about 3 mm, about 3.5 mm, about 4 mm, about 4.5 mm, about 5 mm, or about 5.5 mm. In some instances, the circular geometry thermoelectric inner diameter may be at least about 0.5 mm, about 1 mm, about 1.5 mm, about 2 mm, about 2.5 mm, about 3 mm, about 3.5 mm, about 4 mm, about 4.5 mm, or about 5 mm. In some instances, the circular geometry thermoelectric inner diameter 124 may be at most about 1 mm, about 1.5 mm, about 2 mm, about 2.5 mm, about 3 mm, about 3.5 mm, about 4 mm, about 4.5 mm, about 5 mm, or about 5.5 mm.

In some cases, the circular geometry thermoelectric outer diameter 126 may be about 4 mm to about 9.5 mm. In some cases, the circular geometry thermoelectric outer diameter 126 may be about 6.5 mm to about 7 mm, about 6.5 mm to about 7.5 mm, about 6.5 mm to about 8 mm, about 6.5 mm to about 8.5 mm, about 6.5 mm to about 9 mm, about 6.5 mm to about 9.5 mm, about 6.5 mm to about 4 mm, about 6.5 mm to about 4.5 mm, about 6.5 mm to about 5 mm, about 6.5 mm to about 5.5 mm, about 7 mm to about 7.5 mm, about 7 mm to about 8 mm, about 7 mm to about 8.5 mm, about 7 mm to about 9 mm, about 7 mm to about 9.5 mm, about 7 mm to about 4 mm, about 7 mm to about 4.5 mm, about 7 mm to about 5 mm, about 7 mm to about 5.5 mm, about 7.5 mm to about 8 mm, about 7.5 mm to about 8.5 mm, about 7.5 mm to about 9 mm, about 7.5 mm to about 9.5 mm, about 7.5 mm to about 4 mm, about 7.5 mm to about 4.5 mm, about 7.5 mm to about 5 mm, about 7.5 mm to about 5.5 mm, about 8 mm to about 8.5 mm, about 8 mm to about 9 mm, about 8 mm to about 9.5 mm, about 8 mm to about 4 mm, about 8 mm to about 4.5 mm, about 8 mm to about 5 mm, about 8 mm to about 5.5 mm, about 8.5 mm to about 9 mm, about 8.5 mm to about 9.5 mm, about 8.5 mm to about 4 mm, about 8.5 mm to about 4.5 mm, about 8.5 mm to about 5 mm, about 8.5 mm to about 5.5 mm, about 9 mm to about 9.5 mm, about 9 mm to about 4 mm, about 9 mm to about 4.5 mm, about 9 mm to about 5 mm, about 9 mm to about 5.5 mm, about 9.5 mm to about 4 mm, about 9.5 mm to about 4.5 mm, about 9.5 mm to about 5 mm, about 9.5 mm to about 5.5 mm, about 4 mm to about 4.5 mm, about 4 mm to about 5 mm, about 4 mm to about 5.5 mm, about 4.5 mm to about 5 mm, about 4.5 mm to about 5.5 mm, or about 5 mm to about 5.5 mm. In some cases, the circular geometry thermoelectric outer diameter 126 may be about 6.5 mm, about 7 mm, about 7.5 mm, about 8 mm, about 8.5 mm, about 9 mm, about 9.5 mm, about 4 mm, about 4.5 mm, about 5 mm, or about 5.5 mm. In some cases, the circular geometry thermoelectric outer diameter 126 may be at least about 6.5 mm, about 7 mm, about 7.5 mm, about 8 mm, about 8.5 mm, about 9 mm, about 9.5 mm, about 4 mm, about 4.5 mm, or about 5 mm. In some cases, the circular geometry thermoelectric outer diameter 126 may be at most about 7 mm, about 7.5 mm, about 8 mm, about 8.5 mm, about 9 mm, about 9.5 mm, about 4 mm, about 4.5 mm, about 5 mm, or about 5.5 mm.

The one or more thermoelectric modules 100 may provide a temperature profile necessary for one or more polynucleotide synthesis reaction. In some instances, the temperature profile of a first thermoelectric module may differ that of a second thermoelectric module. In some cases, the temperature profile of the first and second thermoelectric module may not influence one another. In some cases, the temperature profile may comprise temperatures of about 10 degrees C. to about 70 degrees C. In some cases, the temperature profile may comprise temperatures of about 10 degrees C. to about 20 degrees C., about 10 degrees C. to about 30 degrees C., about 10 degrees C. to about 40 degrees C., about 10 degrees C. to about 50 degrees C., about 10 degrees C. to about 60 degrees C., about 10 degrees C. to about 70 degrees C., about 20 degrees C. to about 30 degrees C., about 20 degrees C. to about 40 degrees C., about 20 degrees C. to about 50 degrees C., about 20 degrees C. to about 60 degrees C., about 20 degrees C. to about 70 degrees C., about 30 degrees C. to about 40 degrees C., about 30 degrees C. to about 50 degrees C., about 30 degrees C. to about 60 degrees C., about 30 degrees C. to about 70 degrees C., about 40 degrees C. to about 50 degrees C., about 40 degrees C. to about 60 degrees C., about 40 degrees C. to about 70 degrees C., about 50 degrees C. to about 60 degrees C., about 50 degrees C. to about 70 degrees C., or about 60 degrees C. to about 70 degrees C. In some cases, the temperature profile may comprise temperatures of about 10 degrees C., about 20 degrees C., about 30 degrees C., about 40 degrees C., about 50 degrees C., about 60 degrees C., or about 70 degrees C. In some cases, the temperature profile may comprise temperatures of at least about 10 degrees C., about 20 degrees C., about 30 degrees C., about 40 degrees C., about 50 degrees C., or about 60 degrees C. In some cases, the temperature profile may comprise temperatures of at most about 20 degrees C., about 30 degrees C., about 40 degrees C., about 50 degrees C., about 60 degrees C., or about 70 degrees C.

In some instances, the thermoelectric module 100 may comprise solder tabs 108 that may be electrically and mechanically coupled to a thermoelectric printed circuit board assembly (PCBA) 102, as seen in FIG. 1. In some instances, the solder tabs 108, may be electrically coupled to the thermoelectric PCBA via conductive silver epoxy, tin solder, nickel solder, led solder or any combination thereof. In some instances, soldering between the solder tabs 108 and the thermoelectric PCBA 102 may be achieved by a solder gun, solder iron, reflow oven or any combination hereof. In some cases, the thermoelectric module emitted thermal energy may be electrically controlled through the solder tabs 108 in electrical communication with a processor of the thermocycler device.

In some cases, the thermoelectric module 100 may be comprised of a thermoelectric material. In some cases, the thermoelectric material may comprise one or more of bismuth telluride (Bi₂Te₃) alloy, lead telluride (PbTe), silicon-germanium alloy, or any combination thereof.

### Light Sources

Aspects of the thermocycler device disclosed herein may comprise one or more light sources. The light sources may be configured to emit one or more spectra of light to determine the amount of product of the one or more polynucleotide synthesis reactions. In some cases, the emitted light spectra may comprise one or more spectra of ultra-violet (UV), visible, near-infrared (NIR), infrared (IR), or any combination thereof. In some instances, the emitted spectra may comprise wavelengths of about 100 nm to about 1,200 nm. In some instances, the emitted light spectra of the one or more light sources may comprise wavelengths of about 100 nm to about 200 nm, about 100 nm to about 300 nm, about 100 nm to about 400 nm, about 100 nm to about 500 nm, about 100 nm to about 600 nm, about 100 nm to about 700 nm, about 100 nm to about 800 nm, about 100 nm to about 900 nm, about 100 nm to about 1,000 nm, about 100 nm to about 1,100 nm, about 100 nm to about 1,200 nm, about 200 nm to about 300 nm, about 200 nm to about 400 nm, about 200 nm to about 500 nm, about 200 nm to about 600 nm, about 200 nm to about 700 nm, about 200 nm to about 800 nm, about 200 nm to about 900 nm, about 200 nm to about 1,000 nm, about 200 nm to about 1,100 nm, about 200 nm to about 1,200 nm, about 300 nm to about 400 nm, about 300 nm to about 500 nm, about 300 nm to about 600 nm, about 300 nm to about 700 nm, about 300 nm to about 800 nm, about 300 nm to about 900 nm, about 300 nm to about 1,000 nm, about 300 nm to about 1,100 nm, about 300 nm to about 1,200 nm, about 400 nm to about 500 nm, about 400 nm to about 600 nm, about 400 nm to about 700 nm, about 400 nm to about 800 nm, about 400 nm to about 900 nm, about 400 nm to about 1,000 nm, about 400 nm to about 1,100 nm, about 400 nm to about 1,200 nm, about 500 nm to about 600 nm, about 500 nm to about 700 nm, about 500 nm to about 800 nm, about 500 nm to about 900 nm, about 500 nm to about 1,000 nm, about 500 nm to about 1,100 nm, about 500 nm to about 1,200 nm, about 600 nm to about 700 nm, about 600 nm to about 800 nm, about 600 nm to about 900 nm, about 600 nm to about 1,000 nm, about 600 nm to about 1,100 nm, about 600 nm to about 1,200 nm, about 700 nm to about 800 nm, about 700 nm to about 900 nm, about 700 nm to about 1,000 nm, about 700 nm to about 1,100 nm, about 700 nm to about 1,200 nm, about 800 nm to about 900 nm, about 800 nm to about 1,000 nm, about 800 nm to about 1,100 nm, about 800 nm to about 1,200 nm, about 900 nm to about 1,000 nm, about 900 nm to about 1,100 nm, about 900 nm to about 1,200 nm, about 1,000 nm to about 1,100 nm, about 1,000 nm to about 1,200 nm, or about 1,100 nm to about 1,200 nm. In some instances, the emitted light spectra of the one or more light sources may comprise wavelengths of about 100 nm, about 200 nm, about 300 nm, about 400 nm, about 500 nm, about 600 nm, about 700 nm, about 800 nm, about 900 nm, about 1,000 nm, about 1,100 nm, or about 1,200 nm. In some instances, the emitted light spectra of the one or more light sources may comprise wavelength of 100 nm, about 200 nm, about 300 nm, about 400 nm, about 500 nm, about 600 nm, about 700 nm, about 800 nm, about 900 nm, about 1,000 nm, or about 1,100 nm. In some instances, the emitted spectra of the one or more light sources may comprise wavelengths of at most about 200 nm, about 300 nm, about 400 nm, about 500 nm, about 600 nm, about 700 nm, about 800 nm, about 900 nm, about 1,000 nm, about 1,100 nm, or about 1,200 nm.

In some cases, the emitted light spectra may be absorbed by the reagents of the polynucleotide synthesis reaction of the sample vial undergoing the polynucleotide synthesis reaction. In some instances, the absorbed emitted light spectra from the light source may be emitted as one or more of fluorescence, phosphorescence, auto-fluorescence, stimulated emission, or any combination thereof of light from the sample undergoing polynucleotide synthesis. In some cases, the emitted light is from a synthesized polynucleotide. In some cases, the emitted light is from a reacted reagent. In some cases, the emitted light is from an unused reagent. In some cases, the emitted spectra of light of the one or more the sample vials may comprise wavelengths of about 100 nm to about 1,200 nm. In some cases the emitted spectra of light of the one or more the sample vials may comprise wavelengths of about 100 nm to about 200 nm, about 100 nm to about 300 nm, about 100 nm to about 400 nm, about 100 nm to about 500 nm, about 100 nm to about 600 nm, about 100 nm to about 700 nm, about 100 nm to about 800 nm, about 100 nm to about 900 nm, about 100 nm to about 1,000 nm, about 100 nm to about 1,100 nm, about 100 nm to about 1,200 nm, about 200 nm to about 300 nm, about 200 nm to about 400 nm, about 200 nm to about 500 nm, about 200 nm to about 600 nm, about 200 nm to about 700 nm, about 200 nm to about 800 nm, about 200 nm to about 900 nm, about 200 nm to about 1,000 nm, about 200 nm to about 1,100 nm, about 200 nm to about 1,200 nm, about 300 nm to about 400 nm, about 300 nm to about 500 nm, about 300 nm to about 600 nm, about 300 nm to about 700 nm, about 300 nm to about 800 nm, about 300 nm to about 900 nm, about 300 nm to about 1,000 nm, about 300 nm to about 1,100 nm, about 300 nm to about 1,200 nm, about 400 nm to about 500 nm, about 400 nm to about 600 nm, about 400 nm to about 700 nm, about 400 nm to about 800 nm, about 400 nm to about 900 nm, about 400 nm to about 1,000 nm, about 400 nm to about 1,100 nm, about 400 nm to about 1,200 nm, about 500 nm to about 600 nm, about 500 nm to about 700 nm, about 500 nm to about 800 nm, about 500 nm to about 900 nm, about 500 nm to about 1,000 nm, about 500 nm to about 1,100 nm, about 500 nm to about 1,200 nm, about 600 nm to about 700 nm, about 600 nm to about 800 nm, about 600 nm to about 900 nm, about 600 nm to about 1,000 nm, about 600 nm to about 1,100 nm, about 600 nm to about 1,200 nm, about 700 nm to about 800 nm, about 700 nm to about 900 nm, about 700 nm to about 1,000 nm, about 700 nm to about 1,100 nm, about 700 nm to about 1,200 nm, about 800 nm to about 900 nm, about 800 nm to about 1,000 nm, about 800 nm to about 1,100 nm, about 800 nm to about 1,200 nm, about 900 nm to about 1,000 nm, about 900 nm to about 1,100 nm, about 900 nm to about 1,200 nm, about 1,000 nm to about 1,100 nm, about 1,000 nm to about 1,200 nm, or about 1,100 nm to about 1,200 nm. In some cases the emitted spectra of light of the one or more the sample vials may comprise wavelengths of about 100 nm, about 200 nm, about 300 nm, about 400 nm, about 500 nm, about 600 nm, about 700 nm, about 800 nm, about 900 nm, about 1,000 nm, about 1,100 nm, or about 1,200 nm. In some cases the emitted spectra of light of the one or more the sample vials may comprise wavelengths of at least about 100 nm, about 200 nm, about 300 nm, about 400 nm, about 500 nm, about 600 nm, about 700 nm, about 800 nm, about 900 nm, about 1,000 nm, or about 1,100 nm. In some cases the emitted spectra of light of the one or more the sample vials may comprise wavelengths of at most about 200 nm, about 300 nm, about 400 nm, about 500 nm, about 600 nm, about 700 nm, about 800 nm, about 900 nm, about 1,000 nm, about 1,100 nm, or about 1,200 nm.

The light source of the thermocycler device may comprise one or more of a coherent laser, low-coherent laser, surface emitting diode, super luminescent diode (SLD), vertical cavity surface emitting laser (VCSEL), light emitting diode (LED), micro electrical mechanical system swept source laser, Fabry Perot tuned swept source laser, piezo-electric stretched tunable laser, joule heating brag grating tunable laser, silicon photonic ring resonator, or any combination thereof. In some instances, the light source may be electrically coupled to the thermoelectric PCBA to tune the output of the light source via programmed or user intervention commands executed by a processor in electrical communication with the light source. In some cases, the emitted light from the light source may be divergent, convergent, collimated, or any combination thereof.

In some instances, the light source may be optically coupled to the sample vial through a reaction chamber 130 comprising a light guide 138, as seen in FIG. 6B. In some cases, the light source may be optically coupled to the sample vial via a light guide 138 comprising an air gap between the sample vial and the light source. Alternatively or in combination, the emitted light from the light source may be optically coupled to the sample via a light guide that may comprise one or more lenses. In some cases, the one or more lenses may comprise a macro, wide-angle, telephoto, fish-eye, ball, biconvex, converging meniscus, biconcave, plano-convex, plano-concave, diverging meniscus, or aspheric doublet lens, or any combination thereof.

### LED Light Source

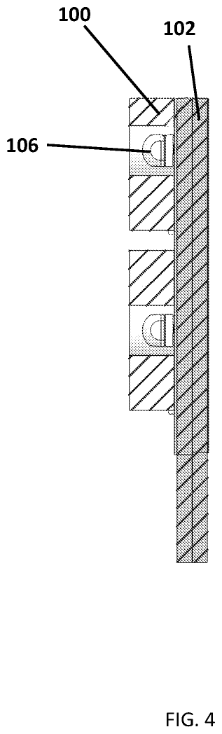

In some cases, the light source may comprise one or more light emitting diode (LED) sources 106, as seen in FIGS. 1-4. In some instances, the one or more LED light sources may be mechanically and electrically coupled to the thermoelectric PCBA 102. In some cases, the one or more LED light sources 106 may be soldered to the thermoelectric PCBA 102. In some instances, the one or more LED light source 108, may be electrically coupled to the thermoelectric PCBA via conductive silver epoxy, tin solder, nickel solder, led solder or any combination thereof. In some instances, soldering between the one or more LED light sources 106 and the thermoelectric PCBA 102 may be achieved by a solder gun, solder iron, reflow oven or any combination hereof. In some cases, the emitted spectra of the LED light source be electrically controlled by a processor of the thermocycler device.

In some cases, the one or more LED light sources 106 may be concentric with respect to the one or more thermoelectric modules 100, as can been in FIGS. 2-4. In some cases, the one or more LED light sources may share a center with the one or more thermoelectric modules 100. The one or more LED light sources 106 may comprise a length 122 and a width 120. In some cases, the length 122 of the one or more LED light sources 106 may be about 0.5 mm to about 5.5 mm. In some cases, the length 122 of the one or more LED 106 light sources may be about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 3.5 mm, about 0.5 mm to about 4 mm, about 0.5 mm to about 5 mm, about 0.5 mm to about 5.5 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 5 mm, about 1 mm to about 5.5 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 1.5 mm to about 5 mm, about 1.5 mm to about 5.5 mm, about 2 mm to about 2.5 mm, about 2 mm to about 3 mm, about 2 mm to about 3.5 mm, about 2 mm to about 4 mm, about 2 mm to about 5 mm, about 2 mm to about 5.5 mm, about 2.5 mm to about 3 mm, about 2.5 mm to about 3.5 mm, about 2.5 mm to about 4 mm, about 2.5 mm to about 5 mm, about 2.5 mm to about 5.5 mm, about 3 mm to about 3.5 mm, about 3 mm to about 4 mm, about 3 mm to about 5 mm, about 3 mm to about 5.5 mm, about 3.5 mm to about 4 mm, about 3.5 mm to about 5 mm, about 3.5 mm to about 5.5 mm, about 4 mm to about 5 mm, about 4 mm to about 5.5 mm, or about 5 mm to about 5.5 mm. In some cases, the length 122 of the one or more LED light sources 106 may be about 0.5 mm, about 1 mm, about 1.5 mm, about 2 mm, about 2.5 mm, about 3 mm, about 3.5 mm, about 4 mm, about 5 mm, or about 5.5 mm. In some cases, the length 122 of the one or more LED light sources 106 may be at least about 0.5 mm, about 1 mm, about 1.5 mm, about 2 mm, about 2.5 mm, about 3 mm, about 3.5 mm, about 4 mm, or about 5 mm. In some cases, the length 122 of the one or more LED light sources 106 may be at most about 1 mm, about 1.5 mm, about 2 mm, about 2.5 mm, about 3 mm, about 3.5 mm, about 4 mm, about 5 mm, or about 5.5 mm.

In some cases, the width 120 of the one or more LED light sources 106 may be about 0.5 mm to about 5.5 mm. In some cases, the width 120 of the one or more LED 106 light sources may be about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 3.5 mm, about 0.5 mm to about 4 mm, about 0.5 mm to about 5 mm, about 0.5 mm to about 5.5 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 5 mm, about 1 mm to about 5.5 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 1.5 mm to about 5 mm, about 1.5 mm to about 5.5 mm, about 2 mm to about 2.5 mm, about 2 mm to about 3 mm, about 2 mm to about 3.5 mm, about 2 mm to about 4 mm, about 2 mm to about 5 mm, about 2 mm to about 5.5 mm, about 2.5 mm to about 3 mm, about 2.5 mm to about 3.5 mm, about 2.5 mm to about 4 mm, about 2.5 mm to about 5 mm, about 2.5 mm to about 5.5 mm, about 3 mm to about 3.5 mm, about 3 mm to about 4 mm, about 3 mm to about 5 mm, about 3 mm to about 5.5 mm, about 3.5 mm to about 4 mm, about 3.5 mm to about 5 mm, about 3.5 mm to about 5.5 mm, about 4 mm to about 5 mm, about 4 mm to about 5.5 mm, or about 5 mm to about 5.5 mm. In some cases, the width 120 of the one or more LED light sources 106 may be about 0.5 mm, about 1 mm, about 1.5 mm, about 2 mm, about 2.5 mm, about 3 mm, about 3.5 mm, about 4 mm, about 5 mm, or about 5.5 mm. In some cases, the width 120 of the one or more LED light sources 106 may be at least about 0.5 mm, about 1 mm, about 1.5 mm, about 2 mm, about 2.5 mm, about 3 mm, about 3.5 mm, about 4 mm, or about 5 mm. In some cases, the width 120 of the one or more LED light sources 106 may be at most about 1 mm, about 1.5 mm, about 2 mm, about 2.5 mm, about 3 mm, about 3.5 mm, about 4 mm, about 5 mm, or about 5.5 mm.

In some instances, the one or more LED light sources 106 may emit a light spectrum with a wavelength spectrum previously described herein. In some cases, the emitted light spectrum of the one or more LED light sources 106 may comprise an output power of about 10 milliwatt (mW) to about 200 mW. In some cases, the emitted light spectrum of the one or more LED light sources 106 may comprise an output power of about 10 mW to about 20 mW, about 10 mW to about 40 mW, about 10 mW to about 60 mW, about 10 mW to about 80 mW, about 10 mW to about 100 mW, about 10 mW to about 120 mW, about 10 mW to about 140 mW, about 10 mW to about 160 mW, about 10 mW to about 180 mW, about 10 mW to about 200 mW, about 20 mW to about 40 mW, about 20 mW to about 60 mW, about 20 mW to about 80 mW, about 20 mW to about 100 mW, about 20 mW to about 120 mW, about 20 mW to about 140 mW, about 20 mW to about 160 mW, about 20 mW to about 180 mW, about 20 mW to about 200 mW, about 40 mW to about 60 mW, about 40 mW to about 80 mW, about 40 mW to about 100 mW, about 40 mW to about 120 mW, about 40 mW to about 140 mW, about 40 mW to about 160 mW, about 40 mW to about 180 mW, about 40 mW to about 200 mW, about 60 mW to about 80 mW, about 60 mW to about 100 mW, about 60 mW to about 120 mW, about 60 mW to about 140 mW, about 60 mW to about 160 mW, about 60 mW to about 180 mW, about 60 mW to about 200 mW, about 80 mW to about 100 mW, about 80 mW to about 120 mW, about 80 mW to about 140 mW, about 80 mW to about 160 mW, about 80 mW to about 180 mW, about 80 mW to about 200 mW, about 100 mW to about 120 mW, about 100 mW to about 140 mW, about 100 mW to about 160 mW, about 100 mW to about 180 mW, about 100 mW to about 200 mW, about 120 mW to about 140 mW, about 120 mW to about 160 mW, about 120 mW to about 180 mW, about 120 mW to about 200 mW, about 140 mW to about 160 mW, about 140 mW to about 180 mW, about 140 mW to about 200 mW, about 160 mW to about 180 mW, about 160 mW to about 200 mW, or about 180 mW to about 200 mW. In some cases, the emitted light spectrum of the one or more LED light sources 106 may comprise an output power of about 10 mW, about 20 mW, about 40 mW, about 60 mW, about 80 mW, about 100 mW, about 120 mW, about 140 mW, about 160 mW, about 180 mW, or about 200 mW. In some cases, the emitted light spectrum of the one or more LED light sources 106 may comprise an output power of at least about 10 mW, about 20 mW, about 40 mW, about 60 mW, about 80 mW, about 100 mW, about 120 mW, about 140 mW, about 160 mW, or about 180 mW. In some cases, the emitted light spectrum of the one or more LED light sources 106 may comprise an output power of at most about 20 mW, about 40 mW, about 60 mW, about 80 mW, about 100 mW, about 120 mW, about 140 mW, about 160 mW, about 180 mW, or about 200 mW.

In some cases, the one or more LED sources 106 may comprise an emitted light field of view of about 10 degrees to about 120 degrees. In some cases, the one or more LED sources 106 may comprise an emitted light field of view of about 10 degrees to about 20 degrees, about 10 degrees to about 30 degrees, about 10 degrees to about 40 degrees, about 10 degrees to about 50 degrees, about 10 degrees to about 60 degrees, about 10 degrees to about 70 degrees, about 10 degrees to about 80 degrees, about 10 degrees to about 90 degrees, about 10 degrees to about 100 degrees, about 10 degrees to about 110 degrees, about 10 degrees to about 120 degrees, about 20 degrees to about 30 degrees, about 20 degrees to about 40 degrees, about 20 degrees to about 50 degrees, about 20 degrees to about 60 degrees, about 20 degrees to about 70 degrees, about 20 degrees to about 80 degrees, about 20 degrees to about 90 degrees, about 20 degrees to about 100 degrees, about 20 degrees to about 110 degrees, about 20 degrees to about 120 degrees, about 30 degrees to about 40 degrees, about 30 degrees to about 50 degrees, about 30 degrees to about 60 degrees, about 30 degrees to about 70 degrees, about 30 degrees to about 80 degrees, about 30 degrees to about 90 degrees, about 30 degrees to about 100 degrees, about 30 degrees to about 110 degrees, about 30 degrees to about 120 degrees, about 40 degrees to about 50 degrees, about 40 degrees to about 60 degrees, about 40 degrees to about 70 degrees, about 40 degrees to about 80 degrees, about 40 degrees to about 90 degrees, about 40 degrees to about 100 degrees, about 40 degrees to about 110 degrees, about 40 degrees to about 120 degrees, about 50 degrees to about 60 degrees, about 50 degrees to about 70 degrees, about 50 degrees to about 80 degrees, about 50 degrees to about 90 degrees, about 50 degrees to about 100 degrees, about 50 degrees to about 110 degrees, about 50 degrees to about 120 degrees, about 60 degrees to about 70 degrees, about 60 degrees to about 80 degrees, about 60 degrees to about 90 degrees, about 60 degrees to about 100 degrees, about 60 degrees to about 110 degrees, about 60 degrees to about 120 degrees, about 70 degrees to about 80 degrees, about 70 degrees to about 90 degrees, about 70 degrees to about 100 degrees, about 70 degrees to about 110 degrees, about 70 degrees to about 120 degrees, about 80 degrees to about 90 degrees, about 80 degrees to about 100 degrees, about 80 degrees to about 110 degrees, about 80 degrees to about 120 degrees, about 90 degrees to about 100 degrees, about 90 degrees to about 110 degrees, about 90 degrees to about 120 degrees, about 100 degrees to about 110 degrees, about 100 degrees to about 120 degrees, or about 110 degrees to about 120 degrees. In some cases, the one or more LED sources 106 may comprise an emitted light field of view of about 10 degrees, about 20 degrees, about 30 degrees, about 40 degrees, about 50 degrees, about 60 degrees, about 70 degrees, about 80 degrees, about 90 degrees, about 100 degrees, about 110 degrees, or about 120 degrees. In some cases, the one or more LED sources 106 may comprise an emitted light field of view of at least about 10 degrees, about 20 degrees, about 30 degrees, about 40 degrees, about 50 degrees, about 60 degrees, about 70 degrees, about 80 degrees, about 90 degrees, about 100 degrees, or about 110 degrees. In some cases, the one or more LED sources 106 may comprise an emitted light field of view of at most about 20 degrees, about 30 degrees, about 40 degrees, about 50 degrees, about 60 degrees, about 70 degrees, about 80 degrees, about 90 degrees, about 100 degrees, about 110 degrees, or about 120 degrees.

### Silicon Photonic Array Light Source

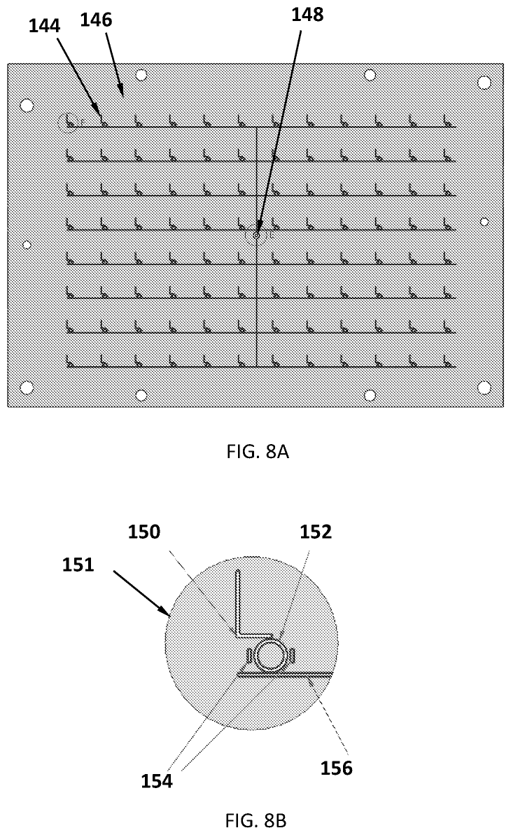

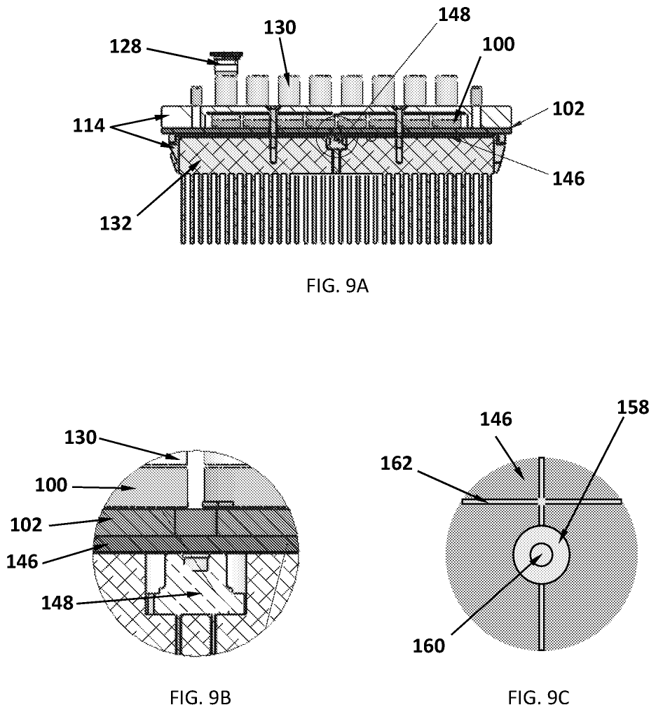

In some cases, the thermocycler device may comprise a light source that may be thermally tunable. In some embodiments, a light beam from the light source is tuned by an thermally tunable optical ring resonator and outputted as a tuned output beam having different properties than the light beam from the light sources. In some instances, the thermocycler device may comprise a silicon photonic array 146 comprising one or more silicon photonic modules 144, a seed light source 148, silicone grating coupler 160, silicone beam splitter 162. Exemplary embodiments of the device comprising a silicon photonic array are seen in FIGS. 8-9. In some cases, the one or more light sources of the thermocycler device may comprise a tunable emission of a silicon photonic array 146, where the silicon photonic array 146 comprises one or more silicon photonic modules 144 comprising a thermally tunable optical ring resonator 151. In some instances, the thermally tunable optical ring resonator 151 may comprise an input silicon wave guide 156, ring resonator cavity 152, one or more thermally tunable elements 154, output silicon wave guide 150, or any combination thereof. In some embodiments, the thermally tunable heater is made of one or more of silicon nitride or silicon carbide or combinations thereof. In some instances, the thermally tunable optical ring resonator 151 of the silicon photonic module 144 may tune an emitted spectrum of light by inducing a change in the refractive index of the thermally tunable optical ring resonator 151.

In some cases, the thermocycler device may comprise a silicon photonic array 146 and a thermoelectric PCBA 102. The silicon photonic array 146 may be configured to be adjacent to, on-top of, or below a thermoelectric PCBA 102, as can been seen in FIGS. 9A-9B. In some instances, the silicon photonic array 146 may be electrically coupled to the thermoelectric PCBA 102. In some instances, the thermoelectric PCBA 102 coupled to the silicon photonic array 146 may comprise a one or more thermoelectric modules 100 electrically coupled to the PCBA as previously described herein. In some cases, the reaction chamber 130, may be mechanically and thermally coupled to the thermoelectric modules 100 via a well holding fixture 114, as seen in FIG. 9A. In some instances, the well holding fixture 114 may comprise a fastener that may fasten the well holding fixture 114 to a heat sink 132. In some cases, the fastener may comprise a hook and clasp, lip and groove, press fit, interference fit, snap fastener, machine screw or any combination thereof.

In some instances, the reaction chamber 130 thermally coupled to the thermoelectric module 100 by the well holding fixture 114 may comprise a light guide 138 as previously described herein. In some cases, the emitted spectrum of light of the one or more silicon photonic modules may couple into a sample vial 128 through the light guide 138.

In some instances, the one or more thermally tunable elements 154 may be tuned in a range of about 10 degrees Kelvin (K) to about 90 degrees K. In some instances, the one or more thermally tunable elements 154 may be tuned in a range of about 10 degrees K to about 20 degrees K, about 10 degrees K to about 30 degrees K, about 10 degrees K to about 40 degrees K, about 10 degrees K to about 50 degrees K, about 10 degrees K to about 60 degrees K, about 10 degrees K to about 70 degrees K, about 10 degrees K to about 80 degrees K, about 10 degrees K to about 90 degrees K, about 20 degrees K to about 30 degrees K, about 20 degrees K to about 40 degrees K, about 20 degrees K to about 50 degrees K, about 20 degrees K to about 60 degrees K, about 20 degrees K to about 70 degrees K, about 20 degrees K to about 80 degrees K, about 20 degrees K to about 90 degrees K, about 30 degrees K to about 40 degrees K, about 30 degrees K to about 50 degrees K, about 30 degrees K to about 60 degrees K, about 30 degrees K to about 70 degrees K, about 30 degrees K to about 80 degrees K, about 30 degrees K to about 90 degrees K, about 40 degrees K to about 50 degrees K, about 40 degrees K to about 60 degrees K, about 40 degrees K to about 70 degrees K, about 40 degrees K to about 80 degrees K, about 40 degrees K to about 90 degrees K, about 50 degrees K to about 60 degrees K, about 50 degrees K to about 70 degrees K, about 50 degrees K to about 80 degrees K, about 50 degrees K to about 90 degrees K, about 60 degrees K to about 70 degrees K, about 60 degrees K to about 80 degrees K, about 60 degrees K to about 90 degrees K, about 70 degrees K to about 80 degrees K, about 70 degrees K to about 90 degrees K, or about 80 degrees K to about 90 degrees K. In some instances, the one or more thermally tunable elements 154 may be tuned in a range of about 10 degrees K, about 20 degrees K, about 30 degrees K, about 40 degrees K, about 50 degrees K, about 60 degrees K, about 70 degrees K, about 80 degrees K, or about 90 degrees K. In some instances, the one or more thermally tunable elements 154 may be tuned in a range of at least about 10 degrees K, about 20 degrees K, about 30 degrees K, about 40 degrees K, about 50 degrees K, about 60 degrees K, about 70 degrees K, or about 80 degrees K. In some instances, the one or more thermally tunable elements 154 may be tuned in a range of at most about 20 degrees K, about 30 degrees K, about 40 degrees K, about 50 degrees K, about 60 degrees K, about 70 degrees K, about 80 degrees K, or about 90 degrees K.

In some cases, the one or more silicon photonic modules 144 may be optically coupled to the seed light source 148 through the silicon photonic array comprising a silicon grating coupler 160, a silicon beam splitter 162, or any combination thereof, as seen in FIG. 9C. In some instances, the seed light source 148 may be optically coupled to a silicon grating coupler 160 with an orthogonal incidence plane with respect to the planar silicon grating coupler 160. The optically coupled light may then be split equally to the one or more silicon photonic modules 144 by the silicon beam splitter 162 comprising a thermally tunable optical ring resonator 151. In some embodiments, the light is split equally to one or more silicon photonic modules by the silicon beam splitter. In some embodiments, the light is split unevenly, directing more light to one or more silicon photonic modules. In some embodiments, the light is split to provide one or more silicon photonic module with at least 10%, 20%, 30%, 40%, 50%, 60%, 70%, 80%, 90%, or 100% more light than the photonic module receiving less light.

In some cases, the seed light source 148 may comprise a coherent laser, low-coherent laser, surface emitting diode, super luminescent diode (SLD), vertical cavity surface emitting laser (VCSEL), light emitting diode (LED), micro electrical mechanical system swept source laser, Fabry Perot tuned swept source laser, piezo-electric stretched tunable laser, joule heating brag grating tunable laser, silicon photonic ring resonator or any combination thereof. The seed light source 148 may comprise an emitted spectrum of about 100 nm to about 200 nm, about 100 nm to about 300 nm, about 100 nm to about 400 nm, about 100 nm to about 500 nm, about 100 nm to about 600 nm, about 100 nm to about 700 nm, about 100 nm to about 800 nm, about 100 nm to about 900 nm, about 100 nm to about 1,000 nm, about 100 nm to about 1,100 nm, about 100 nm to about 1,200 nm, about 200 nm to about 300 nm, about 200 nm to about 400 nm, about 200 nm to about 500 nm, about 200 nm to about 600 nm, about 200 nm to about 700 nm, about 200 nm to about 800 nm, about 200 nm to about 900 nm, about 200 nm to about 1,000 nm, about 200 nm to about 1,100 nm, about 200 nm to about 1,200 nm, about 300 nm to about 400 nm, about 300 nm to about 500 nm, about 300 nm to about 600 nm, about 300 nm to about 700 nm, about 300 nm to about 800 nm, about 300 nm to about 900 nm, about 300 nm to about 1,000 nm, about 300 nm to about 1,100 nm, about 300 nm to about 1,200 nm, about 400 nm to about 500 nm, about 400 nm to about 600 nm, about 400 nm to about 700 nm, about 400 nm to about 800 nm, about 400 nm to about 900 nm, about 400 nm to about 1,000 nm, about 400 nm to about 1,100 nm, about 400 nm to about 1,200 nm, about 500 nm to about 600 nm, about 500 nm to about 700 nm, about 500 nm to about 800 nm, about 500 nm to about 900 nm, about 500 nm to about 1,000 nm, about 500 nm to about 1,100 nm, about 500 nm to about 1,200 nm, about 600 nm to about 700 nm, about 600 nm to about 800 nm, about 600 nm to about 900 nm, about 600 nm to about 1,000 nm, about 600 nm to about 1,100 nm, about 600 nm to about 1,200 nm, about 700 nm to about 800 nm, about 700 nm to about 900 nm, about 700 nm to about 1,000 nm, about 700 nm to about 1,100 nm, about 700 nm to about 1,200 nm, about 800 nm to about 900 nm, about 800 nm to about 1,000 nm, about 800 nm to about 1,100 nm, about 800 nm to about 1,200 nm, about 900 nm to about 1,000 nm, about 900 nm to about 1,100 nm, about 900 nm to about 1,200 nm, about 1,000 nm to about 1,100 nm, about 1,000 nm to about 1,200 nm, or about 1,100 nm to about 1,200 nm. The seed light source 148 may comprise an emitted spectrum of about 100 nm, about 200 nm, about 300 nm, about 400 nm, about 500 nm, about 600 nm, about 700 nm, about 800 nm, about 900 nm, about 1,000 nm, about 1,100 nm, or about 1,200 nm. The seed light source 148 may comprise an emitted spectrum of at least about 100 nm, about 200 nm, about 300 nm, about 400 nm, about 500 nm, about 600 nm, about 700 nm, about 800 nm, about 900 nm, about 1,000 nm, or about 1,100 nm. The seed light source 148 may comprise an emitted spectrum of at most about 200 nm, about 300 nm, about 400 nm, about 500 nm, about 600 nm, about 700 nm, about 800 nm, about 900 nm, about 1,000 nm, about 1,100 nm, or about 1,200 nm.

In some cases, one or more thermally tunable silicon ring resonators 151 may receive a seed light source at one or more first wavelengths from the seed light source 148 and emit a second one or more wavelengths. In some cases, the first and second one or more wavelengths received and emitted by the one or more thermally tunable silicon ring resonator light sources 151 may differ by about 1 nm to about 12 nm. In some cases, the first and second one or more wavelengths received and emitted by the one or more thermally tunable silicon ring resonator light sources 151 may differ by about 1 nm to about 2 nm, about 1 nm to about 3 nm, about 1 nm to about 4 nm, about 1 nm to about 5 nm, about 1 nm to about 6 nm, about 1 nm to about 7 nm, about 1 nm to about 8 nm, about 1 nm to about 9 nm, about 1 nm to about 10 nm, about 1 nm to about 11 nm, about 1 nm to about 12 nm, about 2 nm to about 3 nm, about 2 nm to about 4 nm, about 2 nm to about 5 nm, about 2 nm to about 6 nm, about 2 nm to about 7 nm, about 2 nm to about 8 nm, about 2 nm to about 9 nm, about 2 nm to about 10 nm, about 2 nm to about 11 nm, about 2 nm to about 12 nm, about 3 nm to about 4 nm, about 3 nm to about 5 nm, about 3 nm to about 6 nm, about 3 nm to about 7 nm, about 3 nm to about 8 nm, about 3 nm to about 9 nm, about 3 nm to about 10 nm, about 3 nm to about 11 nm, about 3 nm to about 12 nm, about 4 nm to about 5 nm, about 4 nm to about 6 nm, about 4 nm to about 7 nm, about 4 nm to about 8 nm, about 4 nm to about 9 nm, about 4 nm to about 10 nm, about 4 nm to about 11 nm, about 4 nm to about 12 nm, about 5 nm to about 6 nm, about 5 nm to about 7 nm, about 5 nm to about 8 nm, about 5 nm to about 9 nm, about 5 nm to about 10 nm, about 5 nm to about 11 nm, about 5 nm to about 12 nm, about 6 nm to about 7 nm, about 6 nm to about 8 nm, about 6 nm to about 9 nm, about 6 nm to about 10 nm, about 6 nm to about 11 nm, about 6 nm to about 12 nm, about 7 nm to about 8 nm, about 7 nm to about 9 nm, about 7 nm to about 10 nm, about 7 nm to about 11 nm, about 7 nm to about 12 nm, about 8 nm to about 9 nm, about 8 nm to about 10 nm, about 8 nm to about 11 nm, about 8 nm to about 12 nm, about 9 nm to about 10 nm, about 9 nm to about 11 nm, about 9 nm to about 12 nm, about 10 nm to about 11 nm, about 10 nm to about 12 nm, or about 11 nm to about 12 nm. In some cases, the first and second one or more wavelengths received and emitted by the one or more thermally tunable silicon ring resonator light sources 151 may differ by about 1 nm, about 2 nm, about 3 nm, about 4 nm, about 5 nm, about 6 nm, about 7 nm, about 8 nm, about 9 nm, about 10 nm, about 11 nm, or about 12 nm. In some cases, the first and second one or more wavelengths received and emitted by the one or more thermally tunable silicon ring resonator light sources 151 may differ by at least about 1 nm, about 2 nm, about 3 nm, about 4 nm, about 5 nm, about 6 nm, about 7 nm, about 8 nm, about 9 nm, about 10 nm, or about 11 nm. In some cases, the first and second one or more wavelengths received and emitted by the one or more thermally tunable silicon ring resonator light sources 151 may differ by at most about 2 nm, about 3 nm, about 4 nm, about 5 nm, about 6 nm, about 7 nm, about 8 nm, about 9 nm, about 10 nm, about 11 nm, or about 12 nm.

### Detection Module

In some instances, the thermocycler device may comprise one or more detection modules 141 optically coupled to the emitted light of the one or more sample vials 128. An exemplary embodiment of the detection module is seen in FIG. 7. In some cases, the one or more detection modules may comprise one or more sensors 140, tunable filters 142 or any combination thereof. In some cases, the one or more detection modules 141 may be electrically coupled to a processor. In some cases, the processor may be configured to read and transmit sensor 140 data, initiate sensor data acquisition, modify a tunable filter 142 filtering parameter, or any combination thereof. In some instances, the detection module may be configured to detect the emitted light from the sample vial 128. In some cases, one or more parameters of the detection module may be adjusted to improve performance of the devices, systems, and methods provided herein. In some cases, the parameters include but are not limited to ISO, integration period, pixel pitch, or quantum efficiency. In some cases, the emitted light from the sample vial may comprise fluorescence, phosphorescence, stimulated emission, auto-fluorescence, fluorescence lifetime or any combination thereof. Alternatively or in combination, the detection module may be configured to detect thermal infrared energy of the sample vial 128 to measure the temperature of the sample vial. In some instances, the detection module may be configured to identify sample vial indicator 104, described elsewhere herein.

In some cases, the sensor comprises one or more of a two-dimensional CMOS, two-dimensional CCD, linear array CMOS, linear array CCD, avalanche photodiode, single photodiode, or balanced photodetector sensor, or any combination thereof. In some instances, the sensor 140 may be sensitive to light spectra of ultra-violet, visible, near-infrared (NIR), infrared (IR), or any combination thereof. In some instances, the one or more sensors 140 may detect wavelengths of about 100 nm to about 1,200 nm. In some instances, the one or more sensors 140 may detect wavelengths of about 100 nm to about 200 nm, about 100 nm to about 300 nm, about 100 nm to about 400 nm, about 100 nm to about 500 nm, about 100 nm to about 600 nm, about 100 nm to about 700 nm, about 100 nm to about 800 nm, about 100 nm to about 900 nm, about 100 nm to about 1,000 nm, about 100 nm to about 1,100 nm, about 100 nm to about 1,200 nm, about 200 nm to about 300 nm, about 200 nm to about 400 nm, about 200 nm to about 500 nm, about 200 nm to about 600 nm, about 200 nm to about 700 nm, about 200 nm to about 800 nm, about 200 nm to about 900 nm, about 200 nm to about 1,000 nm, about 200 nm to about 1,100 nm, about 200 nm to about 1,200 nm, about 300 nm to about 400 nm, about 300 nm to about 500 nm, about 300 nm to about 600 nm, about 300 nm to about 700 nm, about 300 nm to about 800 nm, about 300 nm to about 900 nm, about 300 nm to about 1,000 nm, about 300 nm to about 1,100 nm, about 300 nm to about 1,200 nm, about 400 nm to about 500 nm, about 400 nm to about 600 nm, about 400 nm to about 700 nm, about 400 nm to about 800 nm, about 400 nm to about 900 nm, about 400 nm to about 1,000 nm, about 400 nm to about 1,100 nm, about 400 nm to about 1,200 nm, about 500 nm to about 600 nm, about 500 nm to about 700 nm, about 500 nm to about 800 nm, about 500 nm to about 900 nm, about 500 nm to about 1,000 nm, about 500 nm to about 1,100 nm, about 500 nm to about 1,200 nm, about 600 nm to about 700 nm, about 600 nm to about 800 nm, about 600 nm to about 900 nm, about 600 nm to about 1,000 nm, about 600 nm to about 1,100 nm, about 600 nm to about 1,200 nm, about 700 nm to about 800 nm, about 700 nm to about 900 nm, about 700 nm to about 1,000 nm, about 700 nm to about 1,100 nm, about 700 nm to about 1,200 nm, about 800 nm to about 900 nm, about 800 nm to about 1,000 nm, about 800 nm to about 1,100 nm, about 800 nm to about 1,200 nm, about 900 nm to about 1,000 nm, about 900 nm to about 1,100 nm, about 900 nm to about 1,200 nm, about 1,000 nm to about 1,100 nm, about 1,000 nm to about 1,200 nm, or about 1,100 nm to about 1,200 nm. In some instances, the one or more sensors 140 may detect wavelengths of about 100 nm, about 200 nm, about 300 nm, about 400 nm, about 500 nm, about 600 nm, about 700 nm, about 800 nm, about 900 nm, about 1,000 nm, about 1,100 nm, or about 1,200 nm. In some instances, the one or more sensors 140 may detect wavelengths of at least about 100 nm, about 200 nm, about 300 nm, about 400 nm, about 500 nm, about 600 nm, about 700 nm, about 800 nm, about 900 nm, about 1,000 nm, or about 1,100 nm. In some instances, the one or more sensors 140 may detect wavelengths of at most about 200 nm, about 300 nm, about 400 nm, about 500 nm, about 600 nm, about 700 nm, about 800 nm, about 900 nm, about 1,000 nm, about 1,100 nm, or about 1,200 nm.

In some cases, the tunable filter may comprise one or more solid-state tunable filters 142. In some instances, the solid-state tunable filter may comprise a liquid crystal tunable filter, an acousto-optic filter, or any combination thereof. In some cases, the one or more solid-state tunable filters may be configured to transmit a first spectra of wavelengths while reflecting a second spectra of wavelengths. In some instances, the first spectra of wavelengths may differ from the second spectra of wavelengths. In some cases, the solid-state tunable filter may be configured to adjust a transmitted spectrum of wavelengths in at real-time imaging speeds. In some instances, real-time imaging speeds may comprise about 25 frames per second (FPS) to about 120 FPS. In some instances, real-time imaging speeds may comprise about 25 FPS to about 30 FPS, about 25 FPS to about 40 FPS, about 25 FPS to about 50 FPS, about 25 FPS to about 60 FPS, about 25 FPS to about 70 FPS, about 25 FPS to about 80 FPS, about 25 FPS to about 90 FPS, about 25 FPS to about 100 FPS, about 25 FPS to about 110 FPS, about 25 FPS to about 120 FPS, about 30 FPS to about 40 FPS, about 30 FPS to about 50 FPS, about 30 FPS to about 60 FPS, about 30 FPS to about 70 FPS, about 30 FPS to about 80 FPS, about 30 FPS to about 90 FPS, about 30 FPS to about 100 FPS, about 30 FPS to about 110 FPS, about 30 FPS to about 120 FPS, about 40 FPS to about 50 FPS, about 40 FPS to about 60 FPS, about 40 FPS to about 70 FPS, about 40 FPS to about 80 FPS, about 40 FPS to about 90 FPS, about 40 FPS to about 100 FPS, about 40 FPS to about 110 FPS, about 40 FPS to about 120 FPS, about 50 FPS to about 60 FPS, about 50 FPS to about 70 FPS, about 50 FPS to about 80 FPS, about 50 FPS to about 90 FPS, about 50 FPS to about 100 FPS, about 50 FPS to about 110 FPS, about 50 FPS to about 120 FPS, about 60 FPS to about 70 FPS, about 60 FPS to about 80 FPS, about 60 FPS to about 90 FPS, about 60 FPS to about 100 FPS, about 60 FPS to about 110 FPS, about 60 FPS to about 120 FPS, about 70 FPS to about 80 FPS, about 70 FPS to about 90 FPS, about 70 FPS to about 100 FPS, about 70 FPS to about 110 FPS, about 70 FPS to about 120 FPS, about 80 FPS to about 90 FPS, about 80 FPS to about 100 FPS, about 80 FPS to about 110 FPS, about 80 FPS to about 120 FPS, about 90 FPS to about 100 FPS, about 90 FPS to about 110 FPS, about 90 FPS to about 120 FPS, about 100 FPS to about 110 FPS, about 100 FPS to about 120 FPS, or about 110 FPS to about 120 FPS. In some instances, real-time imaging speeds may comprise about 25 FPS, about 30 FPS, about 40 FPS, about 50 FPS, about 60 FPS, about 70 FPS, about 80 FPS, about 90 FPS, about 100 FPS, about 110 FPS, or about 120 FPS. In some instances, real-time imaging speeds may comprise at least about 25 FPS, about 30 FPS, about 40 FPS, about 50 FPS, about 60 FPS, about 70 FPS, about 80 FPS, about 90 FPS, about 100 FPS, or about 110 FPS. In some instances, real-time imaging speeds may comprise at most about 30 FPS, about 40 FPS, about 50 FPS, about 60 FPS, about 70 FPS, about 80 FPS, about 90 FPS, about 100 FPS, about 110 FPS, or about 120 FPS.

### Methods of Monitoring of Polynucleotide Synthesis

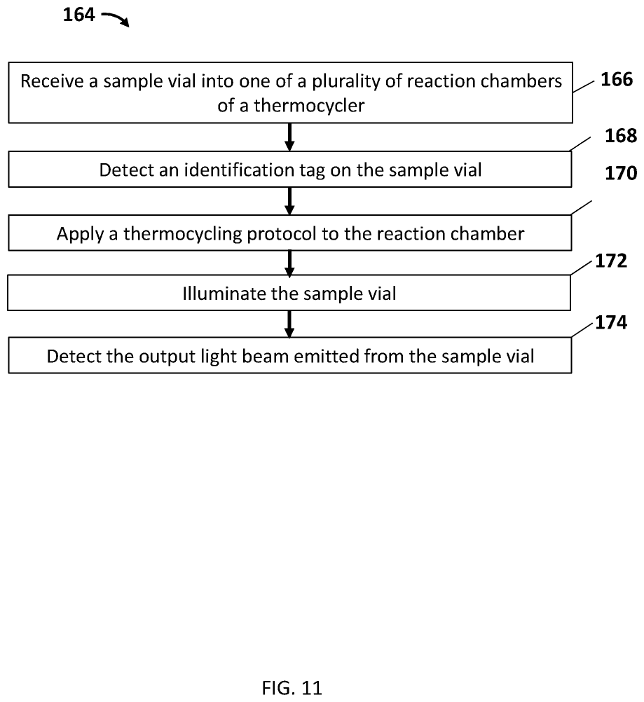

Provided herein are methods of monitoring thermocycling for polynucleotide synthesis of a sample. In some embodiments, the methods of monitoring the polynucleotide synthesis 164, as seen in FIG. 11. In some instances, the method of monitoring the polynucleotide synthesis 164 may comprise the steps of: (a) receiving a sample vial holding a sample 128 into one of a plurality of reaction chambers 130 of a thermocycler 166; (b) detecting by a detection module 141 an identification tag 104 on the sample vial 128, where the identification tag 104 may comprise information of a thermocycling protocol 168; (c) applying the thermocycling protocol to the reaction chamber 130 by a thermoelectric module 100 of the thermocycler, where the thermoelectric module 100 delivers thermal energy to the reaction chamber 130 and the sample vial 128 according to the thermocycling protocol 170; (d) illuminating the sample vial 128 in the reaction chamber 130 with a light beam from a light source (144 or 106) 172; and (e) detecting by the detection module 141 an output light beam emitted from the sample vial 128, where the output light beam comprises the light beam that passed through the sample. In some embodiments, the sample received in step (a) the sample vial 128 may undergo polynucleotide synthesis. In some embodiments, the sample comprises a fluorescent label. In some embodiments, the sample comprises a fluorescently labeled nucleotide probe. Although the steps show a method of a system in accordance with an example, a person of ordinary skill in the art will recognize many variations based on the teaching described herein. The steps may be completed in a different order. Steps may be added or deleted. Some of the steps may comprise sub-steps. Many of the steps may be repeated as often as if beneficial to the platform.

In some cases, the polynucleotide synthesis may be achieved through the use of the devices, systems, and methods described herein. In some instances, the method of polynucleotide synthesis may comprise one or more polynucleotide synthesis reactions to occur with varying start points. In some instances, the method of polynucleotide synthesis may comprise one or more simultaneous polynucleotide reactions comprising one or more temperature profiles. The method of polynucleotide synthesis may comprise one or more quantifiable feedback metrics that may optimize the one or more polynucleotide synthesis reactions. In some embodiments, the information may be gathered from monitoring of the progress of the polynucleotide synthesis, including but not limited to temperature of the sample vial, the lid of the sample vial, and/or the reaction chamber, or concentration, quality, and/or amount of the synthesized polynucleotides in each individual sample vial. In some embodiments, the information gathered from monitoring may be fed back into the device, where the device may decide to change one or more of the polynucleotide synthesis reaction parameters in one or more individual reaction chambers and its associated sample vial. In some embodiments, the reaction parameter that are adjusted include but are not limited to temperature, duration of temperature, applied thermal energy, the wavelength of the light source applied, or intensity of the light source.

In some embodiments, the thermoelectric module comprises a plurality of openings, wherein the light source is oriented to provide a light beam through the plurality of openings of the thermoelectric module. In some embodiments, the thermoelectric module is configured to provide a thermal energy to each the plurality of reaction chambers. In some embodiments, the thermal energy for a first reaction chamber is different from the thermal energy for a second reaction chamber.

In some embodiments, detecting by a detection module an identification tag comprises identifying the identification tag by a system controller and matching the identification tag to the thermocycling protocol in a database. In some embodiments, the identification tag comprises one or more of a quick response (QR) code, barcode, alpha-numeric characters, quantum dots, or radio frequency identification (RFID), or any combination thereof. In some embodiments, the detection module comprises a sensor.

In some embodiments, the method comprises analyzing by a processor the output light beam to determine an amount of synthesized polynucleotides in the sample. In some embodiments, method comprises repeating the steps of illuminating, detecting, and analyzing during polynucleotide synthesis. In some embodiments, method comprises adjusting the tunable filter by the system controller to a target wavelength to image a thermochromic label on the sample vial. In some embodiments, the method comprises adjusting the tunable filter by the system controller to a target near-infrared or infrared spectra to measure a temperature of the reaction chamber. In some embodiments, the method further comprising adjusting the tunable filter by the system controller to a target output wavelength to detect a output light beam from the sample.

In some embodiments, the thermocycling protocol comprises a set of target temperatures for the reaction chamber to reach during the polynucleotide synthesis. In some embodiments, the method comprises applying the thermocycling protocol to the reaction chamber by the thermoelectric module until the detection module detects a color change on the thermochromic label indicating a target temperature. In some embodiments, the method further comprising applying the thermocycling protocol to the reaction chamber by the thermoelectric module until the detection module detects a spectra of near-infrared or infrared emitted by the reaction chamber indicating a target temperature. In some embodiments, the target output wavelength comprises wavelength spectra in the ultra-violet, visible, near-infrared, infrared or any combination thereof.

In some embodiments, the light source comprises a LED. In some embodiments, the LED is a surface mount or through hole LED. In some embodiments, the LED emits ultra violet (UV), visible, near-infrared (NIR), or infrared (IR) radiative energy, or any combination thereof. In some embodiments, the light source comprises a low coherent diode laser, highly coherent diode laser, super luminescent diode (SLD), vertical cavity surface emitting laser (VCSEL) or any combination thereof.

In some embodiments, the detection module comprises a tunable filter, where the thermocycler comprises a system controller is configured to adjust the tunable filter to a target wavelength spectra. In some embodiments, the target wavelength spectra comprise spectra in the ultra-violet, visible, near-infrared, or infrared, or any combination thereof. In some embodiments, the system controller adjusts the tunable filter to a target wavelength range based on the thermocycling protocol. In some embodiments, the method comprises adjusting the tunable filter by the system controller to visible wavelengths to image the identification tag. In some embodiments, the tunable filter is solid state tunable filter. In some embodiments, the tunable filter comprises a liquid crystal tunable filter, or acousto-optic filter, or any combination thereof. In some embodiments, the tunable filter is configured to capture at least IR, visible, and UV wavelengths. In some embodiments, the tunable filter is configured to differentiate between visible and UV light.

In some embodiments, the detection module is configured to image visible and UV light spectra. In some embodiments, the detection module is configured to detect fluorescence, phosphorescence, stimulated emission, auto-fluorescence, fluorescence lifetime emission or any combination thereof. In some embodiments, the sensor for the detection module comprises one or more of a two-dimensional CMOS, two-dimensional CCD, linear array CMOS, linear array CCD, avalanche photodiode, single photodiode, balanced photodetector sensor, or any combination thereof.

In some embodiments, the output light beam comprises one or more of a fluorescence, phosphorescence, stimulated emission, auto-fluorescence, or fluorescence lifetime emission, or any combination thereof. In some embodiments, an intensity of the output light beam is correlated to the amount of the synthesized polynucleotide in the sample. In some embodiments, a wavelength of the output light beam is correlated to the amount of the synthesized polynucleotide in the sample.

In some embodiments, the thermocycler further comprises a silicon photonic array, the silicon photonic array comprising a plurality of silicon photonic modules, each silicon photonic comprising an optical ring resonator. In some embodiments, the optical ring resonator is configured to tune the wavelength of the light beam. In some embodiments, the tuned light beam is detectable by the detection module. In some embodiments, the tuned light beam has a different wavelength from a wavelength of the light source. In some embodiments, the silicon photonic module comprises a grating coupler and a beam splitter, wherein the grating coupler and the beam splitter are configured to direct the light beam from the light source to each of the plurality of optical ring resonators. In some embodiments, the optical ring resonator comprises a thermally tunable optical ring resonator. In some embodiments, the silicon photonic module further comprises an integrated heater, wherein the integrated heater is configured to provide a thermal energy to one of the plurality of thermally tunable optical ring resonators. In some embodiments, the thermal energy provided to one of the plurality of thermally tunable optical ring resonators tunes the light beam.

In some embodiments, the thermocycler comprises a power source, wherein the power source provide power to the thermoelectric module. In some embodiments, the power ranges from about 0W to about 5W. In some embodiments, the thermocycler comprises a system controller that is electrically coupled to the thermoelectric module, the light source, and the detection module.

### Thermocycler Systems

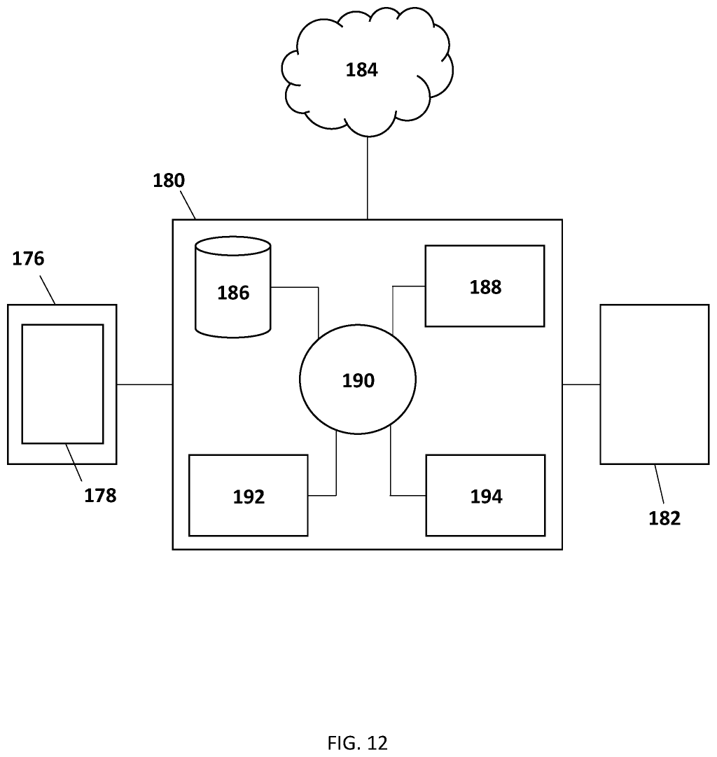

FIG. 12 shows a computer system 180 suitable for implementing a software that may control the thermocycler device and polynucleotide synthesis reactions. The computer system 180 may process various aspects of information of the present disclosure, such as, for example, the identification tag 104 of the sample vial 128. The computer system 180 may be an electronic device. The electronic device may be a mobile electronic device.

The computer system 180 may comprise a central processing unit (CPU, also “processor” and “computer processor” herein) 190, which may be a single core or multi core processor, or a plurality of processor for parallel processing. The computer system 180 may further comprise memory or memory locations 192 (e.g., random-access memory, read-only memory, flash memory), electronic storage unit 186 (e.g., hard disk), communications interface 188 (e.g., network adapter) for communicating with one or more other devices, and peripheral devices 194, such as cache, other memory, data storage and/or electronic display adapters. The memory 192, storage unit 186, interface 188, thermocycler device 182 and peripheral devices 194 are in communication with the CPU 190 through a communication bus (solid lines), such as a motherboard. The storage unit 186 may be a data storage unit (or a data repository) for storing data. The computer system 180 may be operatively coupled to a computer network (“network”) 184 with the aid of the communication interface 188. The network 184 may be the Internet, an internet and/or extranet, or an intranet and/or extranet that is in communication with the Internet. The network 184 may, in some case, be a telecommunication and/or data network. The network 184 may include one or more computer servers, which may enable distributed computing, such as cloud computing. The network 184, in some cases with the aid of the computer system 180, may implement a peer-to-peer network, which may enable devices coupled to the computer system 180 to behave as a client or a server.

The CPU 190 may execute a sequence of machine-readable instructions, which may be embodied in a program or software. The instructions may be directed to the CPU 190, which may subsequently program or otherwise configured the CPU 190 to implement methods of the present disclosure. Examples of operations performed by the CPU 190 may include fetch, decode, execute, and writeback.

The CPU 190 may be part of a circuit, such as an integrated circuit. One or more other components of the system 180 may be included in the circuit. In some cases, the circuit is an application specific integrated circuit (ASIC).

The storage unit 186 may store files, such as drivers, libraries and saved programs. The storage unit 186 may comprise the one or more polynucleotide synthesis reaction temperature profile associated one or more identification tags 104, temporally recorded temperature data of the one or more samples contained in the sample vials 128, temporal light emission intensity of the sample contained in the sample vials 128, or any combination thereof. The computer system 180, in some cases may include one or more additional data storage units that are external to the computer system 501, such as located on a remote server that is in communication with the computer system 180 through an intranet or the internet.

Methods as described herein may be implemented by way of machine (e.g., computer processor) executable code stored on an electronic storage location of the computer device 180, such as, for example, on the memory 192 or electronic storage unit 186. The machine executable or machine-readable code may be provided in the form of software. During use, the code may be executed by the processor 190. In some instances, the code may be retrieved from the storage unit 186 and stored on the memory 192 for ready access by the processor 190. In some instances, the electronic storage unit 186 may be precluded, and machine-executable instructions are stored on memory 192.

The code may be pre-compiled and configured for use with a machine having a processor adapted to execute the code or may be compiled during runtime. The code may be supplied in a programming language that may be selected to enable the code to be executed in a pre-complied or as-compiled fashion.

Aspects of the systems and methods provided herein, such as the computer system 180, may be embodied in programming. Various aspects of the technology may be thought of a “product” or “articles of manufacture” typically in the form of a machine (or processor) executable code and/or associated data that is carried on or embodied in a type of machine readable medium. Machine-executable code may be stored on an electronic storage unit, such memory (e.g., read-only memory, random-access memory, flash memory) or a hard disk. “Storage” type media may include any or all of the tangible memory of a computer, processor the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage’ media, term such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media may include, for example, optical or magnetic disks, such as any of the storage devices in any computer(s) or the like, such as may be used to implement the databases, etc. Volatile storage media include dynamic memory, such as main memory of such a computer platform. Tangible transmission media includes coaxial cables; copper wire and fiber optics, including the wires that comprise a bus within a computer device. Carrier-wave transmission media may take the form of electric or electromagnetic signals, or acoustic or light waves such as those generated during radio frequency (RF) and infrared (IR) data communications. Common forms of computer-readable media therefor include for example: a floppy disk, a flexible disk, hard disk, magnetic tape, any other magnetic medium, a CD-ROM, DVD or DVD-ROM, any other optical medium, punch cards paper tape, any other physical storage medium with pattern of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one more instruction to a processor for execution.

The computer system may include or be in communication with an electronic display 176 that comprises a user interface (UI) 178 for viewing and interfacing with the one or more polynucleotide synthesis reactions measured metrics comprising: temporally recorded temperature data of the one or more samples contained in the sample vials 128, temporal light emission intensity of the sample contained in the sample vials 128, or any combination thereof. Examples of UI's include, without limitation, a graphical user interface (GUI) and web-based user interface.

Methods and systems of the present disclosure can be implemented by way of one or more algorithms and with instructions provided with one or more processors as disclosed herein. An algorithm can be implemented by way of software upon execution by the central processing unit 190.

In some embodiments, the devices, systems, and methods provided herein comprises an optical detection and independent well control, which may enable real-time detection and monitoring of concentrations of synthesized polynucleotides in each well. In some embodiments, the devices, systems, and methods provided herein comprises a thermoelectric module, temperature sensor, a control system, and a custom thermoelectric module, a surface mounted UV LED, a video camera (for detection) above the wells, a solid state tunable filter that is used with the video camera (to filter incident light) and allow a user to differentiate between visible and UV light. For example, visible light is used to identify a sample by QR/barcode as well as to detect the temperature of a vial lid.

These embodiments can provide data, for example reaction rate, melting temperature, final concentration, and other data that can be used for machine learning algorithms which are described and found in other embodiments described in the related provisional patent applications previously referenced and incorporated herein. By using the data in conjunction with the machine learning algorithms, many of these embodiments can lead to optimal PCR conditions and a better understanding of how key input variables affect PCR performance.

Along with the ability to control each well independently in these embodiments, end users can indicate the final concentration of the DNA they would like to end up with in each well and when each well reaches the desired one or more concentrations, the system controller stops cycling the well temperature.

In some embodiments, the thermoelectric PCBA (“TEC board”) has a surface mounted LED and custom, “round” thermoelectric modules. In some embodiments, a center placement of the surface mounted UV LED is relative to the round, custom thermoelectric module. In some embodiments, a light path from the UV LED goes through the center cutout of the custom, round thermoelectric module. In some embodiments, a round shape of a custom thermoelectric module and the solder tabs are used to position it and provide electrical connectivity. In some embodiments, the well has its center bored out, and there is a light pipe inside this bored center which provides collimated light from the UV LED positioned on the bottom surface. In some embodiments, the thermoelectric module component may be modified from having a 6 mm square footprint to having an 8 mm round footprint with a 2.50 mm hole in the center. In some embodiments, the solder tabs are used in place of the lead wires so that the solder tabs can be directly soldered to the top of the existing TEC board. In some embodiments, the individual PCR wells have the lower portion bored out so that there is a hole in the bottom of each PCR well. This may allow light from one or more excitation LEDs to illuminate the bottom of each PCR well. In some embodiments, a light pipe or collimating lens included with the LED to reduce scattering may be used.

In some embodiments, the thermoelectric PCB having soldered thermoelectric modules may have donut shaped thermoelectric modules. In some embodiments, a single surface mount LED (diameter˜1.6 mm) may be placed inside the center of each thermoelectric module. In some embodiments, the surface mount LED can either be a single wavelength or multi-model to emit light at a number of wavelengths. In some embodiments, the surface mount LED can have a diameter greater or less than 1.6 mm.

In some embodiments, the devices described herein comprise a camera/CCD, a lens, and a solid state optical light filter located in front of the camera/CCD. In some embodiments, the solid state optical light filter can be used to discern a variety of wavelengths. In some embodiments, a number of different solid state optical light filters may be used.

In some embodiments, reagents comprise one or more of oligonucleotides, dNTP's, enzymes, or a dye, such as SYBR green, or combinations thereof. In some embodiments, the reagents are placed into a consumable sample vial. Any other suitable known reagent known to those skilled in the art may also be used.

### Definitions

The practice of some methods disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

The terms “about” and “approximately” mean within an acceptable error range for the particular value as determined by one of ordinary skill in the art, which will depend in part on how the value is measured or determined, such as the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about,” meaning within an acceptable error range for the particular value, should be assumed.

As used herein, the terms “polynucleotide”, “nucleic acid,” “oligonucleotide,” and “gene” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, isolated DNA of any sequence, cDNA, recombinant polynucleotides, branched polynucleotides, plasmids, vectors, isolated RNA of any sequence, messenger RNA (mRNA), transfer RNA (tRNA), ribosomal RNA (rRNA), short interfering RNA (siRNA), short-hairpin RNA (shRNA), micro-RNA (miRNA), ribozymes, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. Generally, oligonucleotides as used herein are shorter than polynucleotides.

The term “strand,” as used herein, refers to a nucleic acid made up of nucleotides covalently linked together by covalent bonds, e.g., phosphodiester bonds. In a cell, DNA usually exists in a double-stranded form, and as such, has two complementary strands of nucleic acid referred to herein as the “top” and “bottom” strands. In certain cases, complementary strands of a chromosomal region may be referred to as “plus” and “minus” strands, the “first” and “second” strands, the “coding” and “noncoding” strands, the “Watson” and “Crick” strands, or the “sense” and “antisense” strands. The assignment of a strand as being a top or bottom strand is arbitrary and does not imply any particular orientation, function or structure.

A polynucleotide may have a 5′ end and 3′ end, referring to the end-to-end chemical orientation of a single strand of polynucleotide or nucleic acid. In a single strand of linear DNA or RNA, the chemical convention of naming carbon atoms in the nucleotide sugar-ring means that there generally exists a 5′ end which frequently contains a phosphate group attached to the 5′ carbon and a 3′ end which typically is unmodified from the ribose —OH substituent (hydroxyl group). In some cases, a polynucleotide may have a —OH substituent or a hydroxyl group at a 5′ end and —P group or phosphate group at a 3′ end. A phosphate group attached to the 5′-end permits ligation of two nucleotides, e.g., the covalent binding of a 5′-phosphate to the 3′-hydroxyl group of another nucleotide, to form a phosphodiester bond. Removal of the 5′-phosphate may inhibit or prevent ligation. The 3′-hydroxyl group is also important as it is joined to the 5′-phosphate in ligation.

The term “primer,” as used herein, generally refers to an oligonucleotide, either natural or synthetic, that is capable, upon forming a duplex with a nucleic acid molecule template, of acting as a point of initiation of nucleic acid synthesis and being extended from its 3′ end along the template so that an extended duplex is formed. The sequence of nucleotides added during the extension reaction may be determined by the sequence of the template nucleic acid molecule. Usually primers are extended by a DNA polymerase. Sometimes primers are extended by a reverse transcriptase. Primers are generally of a length compatible with their use in synthesis of primer extension products, and usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

The terms “isolated” and “isolating,” with reference to a nucleic acid molecule or nucleic acid molecule complex generally refer to a preparation of the substance (e.g., nucleic acid molecule, nucleic acid molecule complex, extension products thereof) devoid of at least some of the other components that may also be present where the substance or a similar substance naturally occurs or is initially obtained from (e.g., a biological sample, a sample reaction volume, e.g., a synthesis reaction volume, etc). For example, an isolated substance may be prepared using a purification technique to enrich it from a source mixture. Enrichment can be measured on an absolute basis or in terms of a concentration, for example in terms of weight per volume of solution, molecules per volume of solution, or any other appropriate measure.

The term “gene synthesis,” as used herein, refers to polynucleotide synthesis or polynucleotide assembly. Polynucleotide synthesis refers to the process of covalently linking a nucleotide to another to another nucleotide, an oligonucleotide to another oligonucleotide, or a nucleotide to an oligonucleotide to generate a strand of nucleic acids, oligonucleotides, or polynucleotides.

As used herein, the terms “gene of interest” and “polynucleotide of interest” are used interchangeably. The terms mean that the sequence of the polynucleotide is known and chosen before synthesis or assembly of the polynucleotide product.

As used herein, the terms “well” and “chamber” are used interchangeably. A well refers to a container capable of holding reagents for the polynucleotide synthesis.

### EXAMPLES

### Example 1. Exemplary Device for Polynucleotide Synthesis

Provided herein is an exemplary device and system suitable for polynucleotide synthesis with the following instrument specification (Table 1). The device is designed to independently control each well with accuracy and simplicity. The device can independently control various settings of the 96 wells, including but not limited to temperature settings. The device can integrate lab automation using an automated lid and API. The device is designed to have a web-based software that provides an easy-to-use interface and traceability of the steps within any single well. The web-based software also provides enhanced collaboration among users, who can be local to the device or remote from the device.

TABLE 1
Device Specifications

| Block Format | 96-well microplate, 0.2 mL; | |
|---|---|---|
| | 8-well strips | |
| Max heating rate (well) | 3° | C./s |
| Max cooling rate (well) | 2.5° | C./s |
| Temperature accuracy | ±0.1° | C. |
| Temperature range | 0-100° | C. |
| Maximum temperature difference | 98° | C. |
| (well to well) | | |
| Dimensions (H × W × D) | 40 cm × 40 cm × 30 cm | |
| Weight | 14 | kg |
| Volume range | 10-100 | μL |

| | |
|---|---|
| Display | 8-inch color TFT LCD |
| Power | 100-240 V, 50-60 Hz, max. 700 W |
| Data connectivity | Cloud or mobile via Ethernet, |
| | WiFi or USB |
| Protocol Storage | Unlimited with web app |
| Number of users | Unlimited with web app |
| Lid pressure | Up to 27 kg |
| Integration with automation | Via API |

### Example 2. Exemplary Thermocylcling Device—Multitherm

Provided herein is an exemplary device for polynucleotide synthesis. The device comprises a 96 well thermocycler with individual well control. The device is automatable and is controlled via protocols stored on web-app.

### Example 3. Datatherm

Provided herein is an exemplary system comprising a recommendation engine for PCR optimization and design of experiment (DOE). The recommendation engine provides recommendation on which reagents at what concentrations and volumes to use in the synthesis protocol.

### Example 4. Exemplary Thermocylcling Device—Minitherm

Provided herein is an exemplary device for polynucleotide synthesis. The device comprises a miniature thermocycler that is capable of thermal control and monitoring of individual wells. The device is small enough to be used on a bench top, modular as to include additional channels, and has a low cost. The device is a modular device that can be powered over USB. The device can be controlled remotely or by USB.

### Example 5. Exemplary System—Liquitherm/Datatherm

Provided herein is an exemplary system comprising a microfluidic liquid handling technology that is reconfigurable. The system comprises a liquid handling robot and has in-situ sensing capabilities to monitor conditions. The system can interface with a miniature thermocycler as described in Example 4. The system can be used to automatically monitor and control liquid handling workflows and variables to accommodate the desired biological product and application.

### Example 6. Exemplary Device, Method, and System

Provided herein is an exemplary method comprising chip-based quality control methods. The method comprises UV/Vis, electrophoresis, and next generation sequencing methods. Also provided herein is an exemplary device that is integrated with the system as described in Example 5 (MiniTherm and LiquiTherm) to form a fully reconfigurable, closed loop workflow (liquid handling, thermocycling and feedback). Also provided herein is an exemplary system using a refined recommendation engine with additional process variables, protocols, and applications.

### Example 7. qPCR Monitoring of Polynucleotide Synthesis

In some embodiments, the method of monitoring the polynucleotide synthesis comprises the following steps:
1) The vial is loaded onto the stack assembly.
2) The camera registers a change in population of the stack assembly and notifies the system controller.
3) The system controller adjusts the optical filter to an emitted wavelength of the QR code.
4) The system controller identifies the sample using the QR code and matches it to the thermocycling protocol in a database.
5) The system controller adjusts the optical filter to the emitted wavelength of thermochromic ink.
6) The system controller applies power to the sample vial to heat the top of the sample until the camera registers the change in color of thermochromic ink matching the desired lid temperature.
7) The system controller loops the optical filter to allow the wavelength from the thermochromic ink or the emission from fluorescent (520 nm) sample to the CCD.
8) During each loop described in steps 1-7, the system controller uses the camera to measure the amount of light from the thermochromic ink or the fluorescence from the sample.

In some embodiments, coherent laser light, which has less noise, may be used to fluoresce each well. In some embodiments, the wavelength of light may be tuned for each well and the end user can use multiple fluorescence tags/dyes.

### EXEMPLARY EMBODIMENTS

Among the exemplary embodiments are:

Embodiment 1 comprises a device for thermocycling for polynucleotide synthesis, comprising: (a) a plurality of reaction chambers, each reaction chamber having a top opening and a bottom opening, wherein the top opening and the bottom opening are on opposite ends; (b) a thermoelectric module, wherein the thermoelectric module is thermally coupled to each of the plurality of reaction chambers; (c) a light source, the light source oriented to provide a light beam through the top opening and the bottom opening of the each of the plurality of reaction chambers; and (d) a detection module, the detection module comprising an imaging module the detection module positioned above the plurality of reaction chambers. Embodiment 2 comprises the device for thermocycling of embodiment 1, wherein the thermoelectric module comprises a plurality of openings, where the light source is oriented to provide a light beam through the plurality of openings of the thermoelectric module. Embodiment 3 comprises the device for thermocycling of any one of embodiments 1-2, wherein the thermoelectric module is configured to provide a thermal energy to each of the plurality of reaction chambers. Embodiment 4 comprises the device for thermocycling of any one of embodiment 1-3, wherein the thermal energy for a first reaction chamber is different from the thermal energy for a second reaction chamber. Embodiment 5 comprises the device for thermocycling of any one of embodiments 1-4, where each of the plurality of reaction chambers is configured to hold a sample container. Embodiment 6 comprises the device for thermocycling of any one of embodiments 1-5, where the sample container comprises a lid comprising an identification tag that is detectable by the detection module. Embodiment 7 comprises the device for thermocycling of any one of embodiments 1-6, wherein the identification tag comprises one or more of a quick response (QR) code, barcode, alpha-numeric characters, quantum dots, or radio frequency identification (RFID). Embodiment 8 comprises the device for thermocycling of any one of embodiments 1-7, where the sample container comprises a lid comprising a temperature indicator that is detectable by the detection module. Embodiment 9 comprises the device for thermocycling of any one of embodiments 1-8, wherein the temperature indicator comprises a thermochromic label. Embodiment 10 comprises the device for thermocycling of any one of embodiments 1-9, wherein the light source comprises a light emitting diode (LED). Embodiment 11 comprises the device for thermocycling of any one of embodiments 1-10, wherein the LED is a surface mount or through hole LED. Embodiment 12 comprises the device for thermocycling of any one of embodiments 1-11, wherein the LED emits ultraviolet(UV), visible, near-infrared (NIR), or infrared (IR) radiative energy, or any combination thereof. Embodiment 13 comprises the device for thermocycling of any one of embodiments 1-12, wherein the detection module further comprises a tunable filter. Embodiment 14 comprises the device for thermocycling of any one of embodiments 1-13, wherein the tunable filter is a solid-state tunable filter. Embodiment 15 comprises the device for thermocycling of any one of embodiments 1-14, where the tunable filter is configured to capture at least IR, visible, and UV wavelengths. Embodiment 16 comprises the device for thermocycling of any one of embodiments 1-15, wherein the tunable filter is configured to differentiate between visible and UV light. Embodiment 17 comprises the device for thermocycling of any one of embodiments 1-16, wherein the tunable filter comprises a liquid crystal tunable filter, or acousto-optic filter, or any combination thereof. Embodiment 18 comprises the device for thermocycling of any one of embodiments 1-17, wherein the detection module is configured to image one or more of visible spectrum, UV light spectrum, or infrared spectrum, or any combination thereof. Embodiment 19 comprises the device for thermocycling of any one of embodiments 1-18, wherein the detection module is configured to detect fluorescence, phosphorescence, stimulated emission, auto-fluorescence, fluorescence lifetime or any combination thereof. Embodiment 20 comprises the device for thermocycling of any one of embodiments 1-19, wherein the light source comprises a tunable emission of a silicon photonic array, wherein the silicon photonic array comprises a plurality of silicon photonic modules, each silicon photonic module comprising an optical ring resonator. Embodiment 21 comprises the device for thermocycling of any one of embodiments 1-20, wherein the optical ring resonator is configured to tune the wavelength of the light source. Embodiment 22 comprises the device for thermocycling of any one of embodiments 1-21, wherein the light source is a low coherent diode laser, highly coherent diode laser, super luminescent diode (SLD), vertical cavity surface emitting laser (VCSEL) or any combination thereof. Embodiment 23 comprises the device for thermocycling of any one of embodiments 1-22, wherein the tuned light beam is detectable by the detection module. Embodiment 24 comprises the device for thermocycling of any one of embodiments 1-23, wherein the tuned light beam has a different wavelength from a wavelength of the light source. Embodiment 25 comprises the device for thermocycling of any one of embodiments 1-24, wherein the silicon photonic module further comprises a grating coupler and a beam splitter, wherein the grating coupler and the beam splitter are configured to direct the light source to each silicon photonic module of the plurality of silicon photonic modules. Embodiment 26 comprises the device for thermocycling of any one of embodiments 1-25, wherein the optical ring resonator comprises a thermally tunable optical ring resonator. Embodiment 27 comprises the device for thermocycling of any one of embodiments 1-26, wherein the silicon photonic module further comprises an integrated heater, wherein the integrated heater is configured to provide a thermal energy to one of the plurality of reaction chambers, and wherein the thermal optical ring resonator is configured to be tuned by the thermal energy. Embodiment 28 comprises the device for thermocycling of any one of embodiments 1-27, wherein the device further comprises a power source, wherein the power source provide power to the thermoelectric module. Embodiment 29 comprises the device for thermocycling of any one of embodiments 1-28, wherein the power ranges from about 0W to about 5W. Embodiment 30 comprises the device for thermocycling of any one of embodiments 1-29, wherein the device is connected to a processor, wherein the processor is electrically coupled to the thermoelectric module, the light source, and the detection module. Embodiment 31 comprises the device for thermocycling of any one of embodiments 1-30, wherein the processor analyzes a detected light beam emitted from the sample in the sample container. Embodiment 32 comprises the device for thermocycling of any one of embodiments 1-31, wherein the detection module comprises a sensor. Embodiment 33 comprises the device for thermocycling of any one of embodiments 1-32, wherein the sensor comprises one or more of a two-dimensional CMOS, two-dimensional CCD, linear array CMOS, linear array CCD, avalanche photodiode, single photodiode, or balanced photodetector sensor, or any combination thereof. Embodiment 34 comprises the device for thermocycling of any one of embodiments 1-33, wherein the detection module comprises an infrared sensor configured to detect a temperature of the reaction chamber. Embodiment 35 comprises the device for thermocycling of any one of embodiments 1-34, wherein a temperature sensor is affixed to a wall of the reaction chamber.

Embodiment 36 comprises a method of monitoring thermocycling for polynucleotide synthesis of a sample, the method comprising: (a) receiving a sample container holding a sample into one of a plurality of reaction chambers of a thermocycler; (b) detecting by a detection module an identification tag on the sample container, wherein the identification tag comprises information of a thermocycling protocol; (c) applying the thermocycling protocol to the reaction chamber by a thermoelectric module of the thermocycler, wherein the thermoelectric module delivers thermal energy to the reaction chamber and the sample container according to the thermocycling protocol; (d) illuminating the sample container in the reaction chamber with a light beam from a light source; and (e) detecting by the detection module an output light beam emitted from the sample container, wherein the output light beam comprises the light beam that passed through the sample. Embodiment 37 comprises the method of monitoring thermocycling of embodiment 36, wherein the sample in the sample container undergoes polynucleotide synthesis. Embodiment 38 comprises the method of monitoring thermocycling of any one of embodiments 36-37, wherein detecting by a detection module an identification tag comprises identifying the identification tag by a system controller and matching the identification tag to the thermocycling protocol in a database. Embodiment 39 comprises the method of monitoring thermocycling of any one of embodiments 36-38, wherein the detection module comprises a sensor. Embodiment 40 comprises the method of monitoring thermocycling of any one of embodiments 36-39, wherein the sensor comprises one or more of a two-dimensional CMOS, two-dimensional CCD, linear array CMOS, linear array CCD, avalanche photodiode, single photodiode, balanced photodetector sensor, or any combination thereof. Embodiment 41 comprises the method of monitoring thermocycling of any one of embodiments 36-40, wherein the identification tag comprises one or more of a quick response (QR) code, barcode, alpha-numeric characters, quantum dots, or radio frequency identification (RFID), or any combination thereof. Embodiment 42 comprises the method of monitoring thermocycling of any one of embodiments 36-41, wherein the method further comprises analyzing by a processor the output light beam to determine an amount of synthesized polynucleotides in the sample. Embodiment 43 comprises the method of monitoring thermocycling of any one of embodiments 36-42, wherein the method comprises repeating the steps of illuminating, detecting, and analyzing during polynucleotide synthesis. Embodiment 44 comprises the method of monitoring thermocycling of any one of embodiments 36-43, wherein the detection module comprises a tunable filter, wherein the thermocycler comprises a system controller that is configured to adjust the tunable filter to a target wavelength spectra. Embodiment 45 comprises the method of monitoring thermocycling of any one of embodiments 36-44, wherein the target wavelength spectra comprise spectra in the ultraviolet, visible, near-infrared, or infrared, or any combination thereof. Embodiment 46 comprises the method of monitoring thermocycling of any one of embodiments 36-45, wherein the system controller adjusts the tunable filter to a target wavelength range based on the thermocycling protocol. Embodiment 47 comprises the method of monitoring thermocycling of any one of embodiments 36-46, the method further comprising adjusting the tunable filter by the system controller to visible wavelengths to image the identification tag. Embodiment 48 comprises the method of monitoring thermocycling of any one of embodiments 36-47, the method further comprising adjusting the tunable filter by the system controller to a target wavelength to image a thermochromic label on the sample container. Embodiment 49 comprises the method of monitoring thermocycling of any one of embodiments 36-48, the method further comprising adjusting the tunable filter by the system controller to a target near-infrared or infrared spectra to measure a temperature of the reaction chamber. Embodiment 50 comprises the method of monitoring thermocycling of any one of embodiment 36-49, the method further comprising applying the thermocycling protocol to the reaction chamber by the thermoelectric module until the detection module detects a color change on the thermochromic label indicating a target temperature. Embodiment 51 comprises the method of monitoring thermocycling of any one of embodiments 36-50, the method further comprising applying the thermocycling protocol to the reaction chamber by the thermoelectric module until the detection module detects a spectra of near-infrared or infrared emitted by the reaction chamber indicating a target temperature. Embodiment 52 comprises the method of monitoring thermocycling of any one of embodiments 36-51, the method further comprising adjusting the tunable filter by the system controller to a target output wavelength to detect an output light beam from the sample. Embodiment 53 comprises the method of monitoring thermocycling of any one of embodiments 36-52, wherein the target output wavelength comprises wavelength spectra in the ultraviolet, visible, near-infrared, infrared or any combination thereof. Embodiment 54 comprises the method of monitoring thermocycling of any one of embodiments 36-53, wherein the sample comprises a fluorescent label. Embodiment 55 comprises the method of monitoring thermocycling of any one of embodiments 36-54, wherein the sample comprises a fluorescently labeled nucleotide probe. Embodiment 56 comprises the method of monitoring thermocycling of any one of embodiments 36-55, wherein the output light beam comprises one or more of a fluorescence, phosphorescence, stimulated emission, auto-fluorescence, or fluorescence lifetime emission, or any combination thereof. Embodiment 57 comprises the method of monitoring thermocycling of any one of embodiments 36-56, wherein an intensity of the output light beam is correlated to the amount of the synthesized polynucleotide in the sample. Embodiment 58 comprises the method of monitoring thermocycling of any one of embodiments 36-57, wherein a wavelength of the output light beam is correlated to the amount of the synthesized polynucleotide in the sample. Embodiment 59 comprises the method of monitoring thermocycling of any one of embodiments 36-58, wherein the thermocycling protocol comprises a set of target temperatures for the reaction chamber to reach during the polynucleotide synthesis. Embodiment 60 comprises the method of monitoring thermocycling of any one of embodiments 36-59, wherein the thermoelectric module comprises a plurality of openings, wherein the light source is oriented to provide a light beam through the plurality of openings of the thermoelectric module. Embodiment 61 comprises the method of monitoring thermocycling of any one of embodiments 36-60, wherein the thermoelectric module is configured to provide a thermal energy to each of the plurality of reaction chambers. Embodiment 62 comprises the method of monitoring thermocycling of any one of embodiments 36-61, wherein the thermal energy for a first reaction chamber is different from the thermal energy for a second reaction chamber. Embodiment 63 comprises the method of monitoring thermocycling of any one of embodiments 36-62, wherein the light source comprises a LED. Embodiment 64 comprises the method of monitoring thermocycling of any one of embodiments 36-63, wherein the LED is a surface mount or through hole LED. Embodiment 65 comprises the method of monitoring thermocycling of any one of embodiments 36-64, wherein the LED emits ultraviolet (UV), visible, near-infrared (NIR), or infrared (IR) radiative energy, or any combination thereof. Embodiment 66 comprises the method of monitoring thermocycling of any one of embodiments 36-65, wherein the tunable filter is a solid-state tunable filter. Embodiment 67 comprises the method of monitoring thermocycling of any one of embodiments 36-66, wherein the tunable filter comprises a liquid crystal tunable filter, or acousto-optic filter, or any combination thereof. Embodiment 68 comprises the method of monitoring thermocycling of any one of embodiments 36-67, wherein the tunable filter is configured to capture at least IR, visible, and UV wavelengths. Embodiment 69 comprises the method of monitoring thermocycling of any one of embodiments 36-68, wherein the tunable filter is configured to differentiate between visible and UV light. Embodiment 70 comprises the method of monitoring thermocycling of any one of embodiments 36-69, wherein the detection module is configured to image visible and UV light spectra. Embodiment 71 comprises the method of monitoring thermocycling of any one of embodiments 36-70, wherein the detection module is configured to detect fluorescence, phosphorescence, stimulated emission, auto-fluorescence, fluorescence lifetime emission or any combination thereof. Embodiment 72 comprises the method of monitoring thermocycling of any one of embodiments 36-71, wherein the light source comprises a low coherent diode laser, high coherent diode laser, super luminescent diode (SLD), vertical cavity surface emitting laser (VCSEL) or any combination thereof. Embodiment 73 comprises the method of monitoring thermocycling of any one of embodiments 36-72, wherein the thermocycler further comprises a silicon photonic array, the silicon photonic array comprising a plurality of silicon photonic modules, each silicon photonic module comprising an optical ring resonator. Embodiment 74 comprises the method of monitoring thermocycling of any one of embodiments 36-73, wherein the optical ring resonator is configured to tune the wavelength of the light beam. Embodiment 75 comprises the method of monitoring thermocycling of any one of embodiments 36-74, wherein the tuned light beam is detectable by the detection module. Embodiment 76 comprises the method of monitoring thermocycling of any one of embodiments 36-75, wherein the tuned light beam has a different wavelength from a wavelength of the light source. Embodiment 77 comprises the method of monitoring thermocycling of any one of embodiments 36-76, wherein the silicon photonic module further comprises a grating coupler and a beam splitter, wherein the grating coupler and the beam splitter are configured to direct the light beam from the light source to each of the plurality of optical ring resonators. Embodiment 78 comprises the method of monitoring thermocycling of any one of embodiments 36-77, wherein the optical ring resonator comprises a thermally tunable optical ring resonator. Embodiment 79 comprises the method of monitoring thermocycling of any one of embodiments 36-78, wherein the silicon photonic module further comprises an integrated heater, wherein the integrated heater is configured to provide a thermal energy to one of the plurality of thermally tunable optical ring resonators. Embodiment 80 comprises the method of monitoring thermocycling of any one of embodiments 36-79, wherein the thermal energy provided to one of the plurality of thermally tunable optical ring resonators tunes the light beam. Embodiment 81 comprises the method of monitoring thermocycling of any one of embodiments 36-80, wherein the thermocycler comprises a power source, wherein the power source provides power to the thermoelectric module. Embodiment 82 comprises the method of monitoring thermocycling of any one of embodiments 36-81, wherein the power ranges from about 0W to about 5W. Embodiment 83 comprises the method of monitoring thermocycling of any one of embodiments 36-82, wherein the thermocycler comprises a system controller that is electrically coupled to the thermoelectric module, the light source, and the detection module.

What is claimed is:

1. A device for thermocycling for polynucleotide synthesis, comprising:
a) a plurality of reaction chambers, each reaction chamber having a top opening and a bottom opening, wherein the top opening and the bottom opening are on opposite ends;
b) a thermoelectric module, wherein the thermoelectric module is thermally coupled to each of the plurality of reaction chambers;
c) a light source, the light source oriented to provide a light beam through the top opening and the bottom opening of the each of the plurality of reaction chambers; and
d) a detection module, the detection module comprising an imaging module the detection module positioned above the plurality of reaction chambers.

2. The device of claim 1, wherein the thermoelectric module comprises a plurality of openings, wherein the light source is oriented to provide a light beam through the plurality of openings of the thermoelectric module.

3. The device of claim 1, wherein the thermoelectric module is configured to provide a thermal energy to each the plurality of reaction chambers.

4. The device of claim 3, wherein the thermal energy for a first reaction chamber is different from the thermal energy for a second reaction chamber.

5. The device of claim 1, wherein each of the plurality of reaction chambers is configured to hold a sample container.

6. The device of claim 5, wherein the sample container comprises a lid comprising an identification tag that is detectable by the detection module.

7. The device of claim 5, wherein the identification tag comprises one or more of a quick response (QR) code, barcode, alpha-numeric characters, quantum dots, or radio frequency identification (RFID).

8. The device of claim 5, wherein the sample container comprises a lid comprising a temperature indicator that is detectable by the detection module.

9. The device of claim 8, wherein the temperature indicator comprises a thermochromic label.

10. The device of claim 1, wherein light source comprises a light emitting diode (LED).

11. The device of claim 10, wherein the LED is a surface mount or through hole LED.

12. The device of claim 10, wherein the LED emits ultra violet (UV), visible, near-infrared (NIR), or infrared (IR) radiative energy, or any combination thereof.

13. The device of claim 1, wherein the detection module further comprises a tunable filter.

14. The device of claim 13, wherein the tunable filter is solid state tunable filter.

15. The device of claim 13, wherein the tunable filter is configured to capture at least IR, visible, and UV wavelengths.

16. The device of claim 13, wherein the tunable filter is configured to differentiate between visible and UV light.

17. The device of claim 16, wherein the tunable filter comprises a liquid crystal tunable filter, or acousto-optic filter, or any combination thereof.

18. The device of claim 13, wherein the detection module is configured to image one or more of visible spectrum, UV light spectrum, or infrared spectrum, or any combination thereof.

19. The device of claim 13, wherein the detection module is configured to detect fluorescence, phosphorescence, stimulated emission, auto-fluorescence, fluorescence lifetime or any combination thereof.

20. The device of claim 1, wherein the light source comprises a tunable emission of a silicon photonic array, wherein the silicon photonic array comprises a plurality of silicon photonic modules, each silicon photonic module comprising an optical ring resonator.

21. The device of claim 20, wherein the optical ring resonator is configured to tune the wavelength of the light source.

22. The device of claim 21, wherein the light source is a low coherent diode laser, highly coherent diode laser, super luminescent diode (SLD), vertical cavity surface emitting laser (VCSEL) or any combination thereof.

23. The device of claim 21, wherein the tuned light beam is detectable by the detection module.

24. The device of claim 21, wherein the tuned light beam has a different wavelength from a wavelength of the light source.

25. The device of claim 20, wherein the silicon photonic module further comprises a grating coupler and a beam splitter, wherein the grating coupler and the beam splitter are configured to direct the light source to each silicon photonic module of the plurality of silicon photonic modules.

26. The device of claim 20, wherein the optical ring resonator comprises a thermally tunable optical ring resonator.

27. The device of claim 20, wherein the silicon photonic module further comprises an integrated heater, wherein the integrated heater is configured to provide a thermal energy to one of the plurality of reaction chambers, and wherein the thermal optical ring resonator is configured to be tuned by the thermal energy.

28. The device of claim 1, wherein the device further comprises a power source, wherein the power source provide power to the thermoelectric module.

29. The device of claim 28, wherein the power ranges from about 0W to about 5W.

30. The device of claim 1, the device connected to a processor, wherein the processor is electrically coupled to the thermoelectric module, the light source, and the detection module.

31. The device of claim 30, wherein the processor analyzes a detected light beam emitted from the sample in the sample vial.

32. The device of claim 1, wherein the detection module comprises a sensor.

33. The device of claim 32, wherein the sensor comprises one or more of a two-dimensional CMOS, two-dimensional CCD, linear array CMOS, linear array CCD, avalanche photodiode, single photodiode, or balanced photodetector sensor, or any combination thereof.

34. The device of claim 1, wherein the detection module comprises an infrared sensor configured to detect a temperature of the reaction chamber.

35. The device of claim 1, wherein a temperature sensor is affixed to a wall of the reaction chamber.
